(19) United States
(12) Patent Application Publication
(10) Pub. No.: US 2025/0009913 A1
(43) Pub. Date: Jan. 9, 2025

(54) COMPOUNDS FOR USE IN NON-COVALENT SCAFFOLD AND METHODS RELATED THERETO

(71) Applicant: NORTH CAROLINA STATE UNIVERSITY, Raleigh, NC (US)

(72) Inventors: Qihui Liu, Raleigh, NC (US); Zhiyuan Wu, Raleigh, NC (US); Jonathan S. Lindsey, Raleigh, NC (US)

(21) Appl. No.: 18/754,452

(22) Filed: Jun. 26, 2024

Related U.S. Application Data
(60) Provisional application No. 63/511,204, filed on Jun. 30, 2023.

(51) Int. Cl. A61K 51/04; A61K 51/08; A61K 51/12

(52) U.S. Cl. CPC A61K 51/0485; A61K 51/088; A61K 51/1213; A61K 2121/00; A61K 2123/00

(57) ABSTRACT

Compounds comprising a tetrapyrrole macrocycle that includes a radionuclide; a hydrogelator attached to the tetrapyrrole macrocycle; a water solubilizing group attached to the hydrogelator; and a cleavage site that is between the hydrogelator and the water solubilizing group are described herein along with their methods of use. Two or more compounds of the present invention may two or more compounds may self-assemble (e.g., aggregate), optionally in vivo.

### RELATED APPLICATION

This application claims priority to and the benefit of U.S. Provisional Patent Application No. 63/511,204, filed Jun. 30, 2023, the disclosure of which is incorporated by reference herein in its entirety.

### GOVERNMENT SUPPORT

This invention was made with government support under grant number 2136700 awarded by the National Science Foundation. The government has certain rights in the invention.

### STATEMENT REGARDING ELECTRONIC FILING OF A SEQUENCE LISTING

A Sequence Listing in XML text format, entitled 5051-1015_ST26, 13,519 bytes in size, generated on Jul. 18, 2024, and filed herewith, is hereby incorporated by reference into the specification for its disclosures.

### FIELD

The present invention concerns radionuclide compounds and methods of use thereof.

### BACKGROUND

Molecular design of self-assembled substances upon enzymatic action under physiological conditions constitutes a new research arena that might be termed “synthetic chemistry in vivo.” The formation of assemblies in vivo is particularly attractive because the resulting scaffold can be exploited to achieve immobilization at desired sites.

However, new compounds and methods are needed.

### SUMMARY

One aspect of the present invention is directed to a compound comprising a tetrapyrrole macrocycle comprising a radionuclide; a hydrogelator attached to the tetrapyrrole macrocycle; a water solubilizing group attached to the hydrogelator; and a cleavage site that is between the hydrogelator and the water solubilizing group.

A further aspect of the present invention is directed to a method of diagnosing a disease or disorder in a subject, the method comprising: administering a compound of the present invention to the subject, thereby diagnosing the disease or disorder in the subject

Another aspect of the present invention is directed to a method of treating a subject in need thereof, the method comprising: administering a compound of the present invention to the subject, thereby treating the subject.

A further aspect of the present invention is directed to a method of aggregating and/or immobilizing a radionuclide in a subject, the method comprising: administering a compound of the present invention to the subject, thereby aggregating and/or immobilizing the radionuclide in the subject. In some embodiments, a cleavage agent (e.g., an enzyme) present in the subject cleaves the compound at the cleavage site, optionally wherein the cleavage agent is a phosphatase, cathepsin, matrix metalloproteinase, serine protease, elastase, urokinase, or urokinase-type plasminogen activator. In some embodiments, two or more compounds of the present invention self-assemble (e.g., aggregate) in the subject, optionally wherein the two or more compounds self-assemble at, and/or around, a tumor in the subject.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 is an illustration of Enzyme-instructed self-assembly (EISA) for use in radiopharmaceutical applications

FIG. 2 is an illustration of an example porphyrin-peptide as the Enzyme-instructed self-assembly (EISA) substrate (Top panel) and an example model molecular design (bottom panel).

FIG. 3 (panel A) Illustration of the cancer targeting porphyrin-peptide as EISA substrate. (panel B) Structure of the designed porphyrin-peptide molecule 5.

FIG. 4 includes absorption spectra of 5 in PBS (containing 0.5% DMSO) at different concentrations. (panel A): 0.10 μM; (panel B): 0.20 μM; (panel C): 0.50 μM; (panel D): 1.0 μM; (panel E): 2.0 μM; (panel F): 5.0 μM. Line curves represent spectra before ALP treatment (−ALP) and after ALP treatment (+ALP). FIG. 5 is an absorption spectra of 5 (10 μM) following ALP treatment and centrifugation with centrifugal devices (MWCO 50 kDa). Samples were centrifuged for 10 min at 10000 rpm. Absorption spectra of the bottom collection (dashed line curve) and top collection (solid line curve) of the 5 sample with ALP treatment after the centrifugation are shown. Samples were diluted by 20-fold before the measurement.

FIG. 6 Absorption spectra of 5 (10 μM) in PBS following treatment of 2 U/mL ALP overnight at 37° C. (solid line curve) and sample after ALP treatment upon addition of DMSO (dashed line curve).

FIG. 7 HPLC traces monitoring phosphorylation of 5 upon treatment with ALP over 8 hours. Signal was detected with a diode array detector at 420 nm. “RT”=retention time.

FIG. 8 Absorption spectra of compound 5′ (panel A) and 4′ (panel B) in DMSO (solid line curve) or PBS (dashed line curve). Concentrations for all samples were 1 μM.

FIG. 9 charts the ratio of the fluorescence intensity of 5′ in PBS to the fluorescence intensity of 5′ in DMSO at different concentrations. Samples in DMSO or PBS were excited at 419 nm or 415 nm, respectively. The maximum fluorescence intensity was recorded at 641 nm for samples in DMSO and 645 nm for samples in PBS. [Ex/Em bandpass=10 nm, integration time=0.05 s].

FIG. 10 is a graph showing reciprocal change in wavelength and concentration of 5: (Dotted line) 0.1 μM with a pathlength of 10 cm; (em-dashed line) 1 μM with a pathlength of 1 cm; (en-dashed line) 10 μM with a pathlength of 0.1 cm; (solid line) 100 μM with a pathlength of 0.01 cm.

### DETAILED DESCRIPTION OF EXAMPLE EMBODIMENTS

The present invention is now described more fully hereinafter with reference to the accompanying drawings, in which embodiments of the invention are shown. This invention may, however, be embodied in many different forms and should not be construed as limited to the embodiments set forth herein; rather these embodiments are provided so that this disclosure will be thorough and complete and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a,” “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

It will also be understood that, as used herein, the terms “example,” “exemplary,” and grammatical variations thereof are intended to refer to non-limiting examples and/or variant embodiments discussed herein, and are not intended to indicate preference for one or more embodiments discussed herein compared to one or more other embodiments.

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

Unless indicated otherwise, nomenclature used to describe chemical groups or moieties as used herein follow the convention where, reading the name from left to right, the point of attachment to the rest of the molecule is at the right hand side of the name. For example, the group “alkylamino” is attached to the rest of the molecule at the amino end, whereas the group “aminoalkyl” is attached to the rest of the molecule at the alkyl end.

Unless indicated otherwise, where a chemical group is described by its chemical formula, including a terminal bond moiety indicated by “—” or “

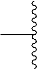

”, it will be understood that the attachment is read from the side in which the bond appears. For example, —O-heteroaryl is attached to the rest of the molecule at the oxygen end.

“Alkyl” as used herein alone or as part of another group, refers to a fully saturated straight or branched chain hydrocarbon containing from 1 to 20 carbon atoms, which can be referred to as a C1-C20 alkyl, and can be substituted or unsubstituted. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, and the like. “Loweralkyl” as used herein, is a subset of alkyl, and, in some embodiments, refers to a saturated straight or branched chain hydrocarbon group containing from 1 to 4 carbon atoms and that can be substituted or unsubstituted. Representative examples of loweralkyl include, but are not limited to, methyl, ethyl, n-propyl, iso-propyl, n-butyl, iso-butyl, tert-butyl, and the like. The term “alkyl” or “loweralkyl” is intended to include both substituted and unsubstituted alkyl or loweralkyl unless otherwise indicated and these groups may be substituted with groups selected from halo, alkyl, haloalkyl, alkenyl, alkynyl, cycloalkyl, cycloalkylalkyl, aryl, arylalkyl, heterocyclo, heterocycloalkyl, heteroaryl, hydroxyl, alkoxy, polyalkoxy such as polyethylene glycol, alkenyloxy, alkynyloxy, haloalkoxy, cycloalkoxy, cycloalkylalkyloxy, aryloxy, arylalkyloxy, heterocyclooxy, heterocycloalkyloxy, mercapto, alkyl-S(O)ₐ, haloalkyl-S(O)ₐ, alkenyl-S(O)ₐ, alkynyl-S(O)ₐ, cycloalkyl-S(O)ₐ, cycloalkylalkyl-S(O)ₐ, aryl-S(O)ₐ, arylalkyl-S(O)ₐ, heterocyclo-S(O)ₐ, heterocycloalkyl-S(O)ₐ, amido, amino, carboxy, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, aminoalkyl, alkylphosphonate, alkylnitrile, acyloxy, ester, amide, sulfonamide, urea, carbamate, carboxylate, alkoxyacylamino, aminoacyloxy, nitro or cyano where a is 0, 1, 2 or 3.

“Alkenyl” as used herein alone or as part of another group, refers to a straight or branched chain hydrocarbon containing from 1 to 20 carbon atoms (or in loweralkenyl 1 to 4 carbon atoms) that includes 1 to 8 double bonds in the normal chain, and can be referred to as a C1-C20 alkenyl. Representative examples of alkenyl include, but are not limited to, vinyl, 2-propenyl, 3-butenyl, 2-butenyl, 4-pentenyl, 3-pentenyl, 2-hexenyl, 3-hexenyl, 2,4-heptadiene, and the like. The term “alkenyl” or “loweralkenyl” is intended to include both substituted and unsubstituted alkenyl or loweralkenyl unless otherwise indicated and these groups may be substituted with groups as described in connection with alkyl and loweralkyl above.

“Alkynyl” as used herein alone or as part of another group, refers to a straight or branched chain hydrocarbon containing from 1 to 20 carbon atoms (or in loweralkynyl 1 to 4 carbon atoms) which include 1 triple bond in the normal chain, and can be referred to as a C1-C20 alkynyl. Representative examples of alkynyl include, but are not limited to, 2-propynyl, 3-butynyl, 2-butynyl, 4-pentynyl, 3-pentynyl, and the like. The term “alkynyl” or “loweralkynyl” is intended to include both substituted and unsubstituted alkynyl or loweralkynyl unless otherwise indicated and these groups may be substituted with the same groups as set forth in connection with alkyl and loweralkyl above.

“Hydrocarbon” as used herein refers to a moiety including carbon and hydrogen that may be substituted or unsubstituted and may be linear or branched. Exemplary hydrocarbons include, but are not limited to, alkyl, alkenyl, alkynyl, cycloalkyl, and aryl groups as defined herein.

“Halo” as used herein refers to any suitable halogen, including —F, —Cl, —Br, and —I.

“Mercapto” as used herein refers to an —SH group.

“Azido” as used herein refers to an —N₃ group.

“Cyano” as used herein refers to a —CN group.

“Hydroxyl” as used herein refers to an —OH group.

“Nitro” as used herein refers to an —NO₂ group.

“Alkoxy” as used herein alone or as part of another group, refers to an alkyl or loweralkyl group, as defined herein, appended to the parent molecular moiety through an oxy group, —O—. Representative examples of alkoxy include, but are not limited to, methoxy, ethoxy, propoxy, 2-propoxy, butoxy, tert-butoxy, pentyloxy, hexyloxy and the like.

“Acyl” as used herein alone or as part of another group refers to a —C(O)R²⁰ group, wherein R²⁰ is an alkyl, alkenyl, alkynyl, cycloalkyl, or aryl.

“Acyloxy” as used herein alone or as part of another group refers to a —OC(O)R²⁰ group, wherein R²⁰ is an alkyl, alkenyl, alkynyl, cycloalkyl, or aryl.

“Haloalkyl” as used herein alone or as part of another group, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, 2-chloro-3-fluoropentyl, and the like.

“Alkylthio” as used herein alone or as part of another group, refers to an alkyl group, as defined herein, appended to the parent molecular moiety through a thio moiety, as defined herein. Representative examples of alkylthio include, but are not limited to, methylthio, ethylthio, tert-butylthio, hexylthio, and the like.

“Cycloalkyl” as used herein alone or as part of another group, refers to a saturated or partially unsaturated cyclic hydrocarbon group containing from 1 to 20 carbon atoms (optionally with a carbon atom replaced in a heterocyclic group as discussed below). A cycloalkyl group may include 0, 1, 2, or more double or triple bonds. A cycloalkyl may be aromatic. Representative examples of cycloalkyl include, cyclopropyl, cyclobutyl, cyclopentyl, cyclohexyl, cycloheptyl, cyclooctyl, and cyclododecyl. These rings may optionally be substituted with additional substituents as described herein such as halo or loweralkyl. The term “cycloalkyl” is generic and intended to include heterocyclic groups as discussed below unless specified otherwise.

“Heterocyclic group” or “heterocyclo” as used herein alone or as part of another group, refers to an aliphatic (e.g., fully or partially saturated heterocyclo) or aromatic heterocyclo (e.g., heteroaryl) ring systems containing at least one heteroatom in a ring. A heterocyclic group may include 1, 2, 3, 4, 5, 6, or more ring systems and examples include monocyclic heterocycles, bicyclic heterocycles, tricyclic heterocycles, and a tetracyclic heterocycles. Monocyclic ring systems are exemplified by any 5 or 6 membered ring containing 1, 2, 3, or 4 heteroatoms independently selected from oxygen, nitrogen and sulfur. The 5 membered ring has from 0-2 double bonds and the 6 membered ring has from 0-3 double bonds. Representative examples of monocyclic ring systems include, but are not limited to, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, and the like. Bicyclic ring systems are exemplified by any of the above monocyclic ring systems fused to an aryl group as defined herein, a cycloalkyl group as defined herein, or another monocyclic ring system as defined herein. Representative examples of bicyclic ring systems include but are not limited to, for example, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and may be optionally substituted with groups selected from halo, alkyl, haloalkyl, alkenyl, alkynyl, cycloalkyl, cycloalkylalkyl, aryl, arylalkyl, heterocyclo, heterocycloalkyl, hydroxyl, alkoxy, alkenyloxy, alkynyloxy, haloalkoxy, cycloalkoxy, cycloalkylalkyloxy, aryloxy, arylalkyloxy, heterocyclooxy, heterocyclolalkyloxy, mercapto, alkyl-S(O)ₘ, haloalkyl-S(O)ₘ, alkenyl-S(O)ₘ, alkynyl-S(O)ₘ, cycloalkyl-S(O)ₘ, cycloalkylalkyl-S(O)ₘ, aryl-S(O)ₘ, arylalkyl-S(O)ₘ, heterocyclo-S(O)ₘ, heterocycloalkyl-S(O)ₘ, amino, alkylamino, alkenylamino, alkynylamino, haloalkylamino, cycloalkylamino, cycloalkylalkylamino, arylamino, arylalkylamino, heterocycloamino, heterocycloalkylamino, disubstituted-amino, acylamino, acyloxy, ester, amide, sulfonamide, urea, alkoxyacylamino, aminoacyloxy, nitro or cyano where m=0, 1, 2 or 3. Examples of tetracyclic heterocycles include, but are not limited to, tetrapyrroles.

“Aryl” as used herein alone or as part of another group, refers to a monocyclic, carbocyclic ring system or a bicyclic, carbocyclic fused ring system having one or more aromatic rings. Representative examples of aryl include, but are not limited to, azulenyl, indanyl, indenyl, naphthyl, phenyl, tetrahydronaphthyl, and the like. The term “aryl” is intended to include both substituted and unsubstituted aryl unless otherwise indicated and these groups may be substituted with the same groups as set forth in connection with alkyl and loweralkyl above.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Amino” as used herein means the radical —NH₂.

“Alkylamino” as used herein alone or as part of another group means the radical —NHR⁵⁰, wherein R⁵⁰ is an alkyl group.

“Ester” as used herein alone or as part of another group refers to a —C(O)OR⁵¹ radical, wherein R⁵¹ is an alkyl, cycloalkyl, alkenyl, alkynyl, or aryl.

“Formyl” as used herein refers to a —C(O) H group.

“Carboxylic acid” as used herein refers to a —C(O) OH group.

“Carboxylic ester” as used herein refers to a —C(O)OR⁵² group, wherein R⁵² is an alkyl, cycloalkyl, alkenyl, alkynyl or aryl.

“Boronate ester” as used herein refers to a —B(O)OR⁵³ group, wherein R⁵³ is an alkyl, cycloalkyl, alkenyl, alkynyl or aryl.

“Phosphate ester” or “phosphoester” as used herein refers to a —P(O)(OR⁵³)₂ group, wherein each R⁵³ is independently an alkyl, cycloalkyl, alkenyl, alkynyl or aryl.

“Sulfoester” as used herein refers to a —S(O)₂(OR⁵³) group, wherein R⁵³ is an alkyl, cycloalkyl, alkenyl, alkynyl or aryl.

“Heteroatom” as used herein refers to O, S or N.

“Pharmaceutically acceptable” as used herein means that the compound, anion, cation, or composition is suitable for administration to a subject to achieve the treatments described herein, without unduly deleterious side effects in light of the severity of the disease and necessity of the treatment.

As used herein, the terms “increase,” “increases,” “increased,” “increasing,” “improve,” “enhance,” and similar terms indicate an elevation in the specified parameter of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduces,” “reduced,” “reduction,” “inhibit,” and similar terms refer to a decrease in the specified parameter of at least about 5%, 10%, 15%, 20%, 25%, 30%, 35%, 40%, 45%, 50%, 55%, 60%, 65%, 70%, 75%, 80%, 85%, 90%, 95%, 97%, or 100% such as compared to another measurable property or quantity (e.g., a control value). In some embodiments, the reduction can result in no or essentially no (i.e., an insignificant amount, e.g., less than about 10% or even 5%) detectable activity or amount.

Provided according to embodiments of the present invention are compounds including a radionuclide and methods immobilizing compounds of the present invention such as by non-covalent association. A compound and/or method of the present invention may be suitable for radionuclide imaging and/or therapy. In some embodiments, the immobilization process can thwart systemic diffusion of a radionuclide such as by assembly (e.g., non-covalent assembly) of the compounds of the present invention and/or enable accumulation of radionuclides in a target location, e.g., a tumor space. The compounds of the present invention comprise a tetrapyrrole macrocycle comprising a radionuclide; a hydrogelator attached to the tetrapyrrole macrocycle; a water solubilizing group attached to the hydrogelator; and a cleavage site that is between the hydrogelator and the water solubilizing group.

A “radionuclide” as used herein refers to a nuclide that is radioactive. In some embodiments, the radionuclide is a radioactive nuclide such as, but not limited to, a copper radionuclide. Exemplary radionuclides include, but are not limited to, ¹²³I, ¹²⁵I, ¹³¹I, ²¹¹At, ⁶⁴Cu, ⁶⁷Cu, ⁴⁴Sc, ⁴⁷Sc, ⁶⁷Ga, ⁶⁸Ga, ⁸⁹Zr, ^{99m}Tc, ¹¹¹In, ¹⁷⁷Lu, ⁵¹Mn, ^{52g}Mn, ^{52m}Mn, ⁸⁶Y, ⁶²Zn, ¹⁸F, and ⁵⁷Co. In some embodiments, a tetrapyrrole macrocycle chelates a radionuclide, including, but are not limited to, a radionuclide selected from ⁴⁴Sc, ⁴⁷Sc, ⁶⁴Cu, ⁶⁷Cu, ⁶⁷Ga, ⁶⁸Ga, ⁸⁹Zr, ^{99m}Tc, ¹¹¹In, ¹⁷⁷Lu, ⁵¹Mn, ^{52g}Mn, ^{52m}Mn, ⁸⁶Y, ⁶²Zn, ¹⁸F, and ⁵⁷Co. In some embodiments, the radionuclide is covalently attached to the tetrapyrrole macrocycle, which may in some embodiments, be an astatine or iodide radionuclide, e.g., ¹²³I, ¹²⁵I, ¹³¹I, ¹⁸F, or ²¹¹At.

In some embodiments, the hydrogelator comprises a peptide (e.g., first peptide). “Hydrogelator” as used herein refers to a moiety that can self-assemble in water to form a three-dimensional network or aggregate, which may encapsulate water. In some embodiments, a hydrogelator can form hydrogel via self-assembly through one or more non-covalent forces such as hydrogen-bonding, π-stacking, electrostatics, and/or hydrophobic forces and/or may form when a target balance of hydrophilicity and hydrophobicity is present. Exemplary hydrogelator sequence may comprise FFY, GGGH (SEQ ID NO:1), ILQINS(SEQ ID NO:2), FKFE (SEQ ID NO:3), FKFEFKFE (SEQ ID NO:4), QQKFQFQFEQQ (SEQ ID NO:5), and/or KKFKFEFEF (SEQ ID NO: 6). In some embodiments, the hydrogelator comprises the sequence FFY, or is Naphthyl (Nap)-FFY. In some embodiments, the hydrogelator comprises the sequence GGGH (SEQ ID NO: 1), or is Palmitoyl-GGGH (SEQ ID NO:1). Exemplary peptides that can be present in a hydrogelator, include, but are not limited to, those described in Table 1.

TABLE 1
Examples of peptide hydrogelators

| Gelator | Feature | Ref |
|---|---|---|
| Nap-FFY | Nanofibril | Shi, et al., Biomacromolecules 2014, |
| | | 15(10), 3559-3568. |
| Palmitoyl-GGGH (SEQ | Nanofibril | Tanaka, et al., J. Am. Chem. Soc. |
| ID NO: 1) | | 2015, 137 (2), 770-775. |
| ILQINS (SEQ ID NO: 2) | Ribbon | Lara, et al., Biomacromolecules |
| | | 2012, 13, 12, 4213-4221 |
| (FKFE)₂ (SEQ ID NOs: 3 | Cofibril | Swanekamp, J. Am. Chem. |
| or 4) | | Soc. 2012, 134, 12, 5556-5559. |
| QQKFQFQFEQQ (SEQ | Nanofiber (random | Rudra, ACS Nano. 2012 Feb 28; |
| ID NO: 5) | coil) | 6(2): 1557-1564. |
| KKFKFEFEF (SEQ ID | nanofibrous hydrogels | Wang et al., Macromol. Biosci. |
| NO: 6) | | 2022, 22 (12), 1-14. |

A compound of the present invention comprises a cleavage site between the hydrogelator and the water solubilizing group. The cleavage site can be at the end of the hydrogelator or within the hydrogelator (e.g., within the structure and/or sequence of the hydrogelator). In some embodiments, a compound of the present invention comprises a peptide (e.g., second peptide) that is separate from the hydrogelator, and the cleavage site is within or at the end of (e.g., after) this peptide. In some embodiments, following cleavage at a cleavage site of a compound of the present invention, the water solubilizing group is cleaved (e.g., removed) from the compound. In some embodiments, following cleavage at a cleavage site of a compound of the present invention, a portion of the cleavage site and the water solubilizing group is cleaved (e.g., removed) from the compound. Following cleavage at the cleavage site, a compound of the present invention that is devoid of the water solubilizing group may be more hydrophobic compared to the hydrophobicity prior to cleavage at the cleavage site. In some embodiments, following cleavage at the cleavage site two more compounds of the present invention may aggregate and/or bind together (e.g., non-covalently). The cleavage site can comprise an enzymatic cleavage site, optionally wherein the cleavage site is an enzymatic cleavage site for a phosphatase, cathepsin, matrix metalloproteinase, serine protease, elastase, urokinase, or urokinase-type plasminogen activator. In some embodiments, the peptide comprising and/or providing the cleave site includes, but is not limited to, GFLG (SEQ ID NO:7), (GG), (SEQ ID NO:8), wherein n is between 1 and 4, CRQAGFSL (SEQ ID NO:9), PLGVR (SEQ ID NO:10), PLGL (SEQ ID NO:11), FFAGLAG (SEQ ID NO: 12), HSSKLQ (SEQ ID NO:13), aFK, wherein a is D-alanine, or NPA. Exemplary tumor-related enzymes, discussed further below, and their corresponding substrates are described in Table 2 and may be utilized to tailor the hydrogelator peptide sequence according to the cancer application.

TABLE 2
Tumor-related enzymes and the corresponding substrates.

| Enzyme | Peptide substrateª | Ref |
|---|---|---|
| Cathepsin B | -GF/L/G- (SEQ ID NO: 7) | Cheng, Adv. Materi. Interfaces, |
| | | 2020, 7 (19), 1-9. |
| Cathepsin X | -(GG)n- (SEQ ID NO: 8) | Li, Carbohydr. Polym. 2014, |
| | | 111, 928-935. |
| Cathepsin E | -CRQAGFSL- (SEQ ID | Li, Biomaterials 2017, 139, 30- |
| | NO: 9) | 38. |
| MMP-2 | -PLG/VR- (SEQ ID | Sun, ACS Appl. Mater. |
| | NO: 10) | Interfaces 2017, 9 (45), 39209- |
| | | 39222 |
| MMP-7 | -PLG/L- (SEQ ID NO: 11) | Tanaka, J. Am. Chem. Soc. |
| | | 2015, 137 (2), 770-775 |
| MMP-9 | -FFAG/LAG- (SEQ ID | Kalafatovic, Biomater. Sci. |
| | NO: 12) | 2015, 3 (2), 246-249 |
| PSA | -HSSKLQ/- (SEQ ID | Denmeade, Cancer Res. 1998, |
| | NO: 13) | 58 (12), 2537-2540 |
| Plasmin | -aFK/- | Chakravarty, J. Med. Chem. |
| | | 1983, 26, 633-638. |
| Neutrophil Elastase | -NPA/- | Dias, Chemistry 2019; 25 |
| | | (7):1696-1700. |

^{a}The peptide sequence is written from N-terminus to C-terminus. “/” represents the cleavage site. Underlined amino acid residues are changeable (e.g., to glycine, proline, valine, leucine, lysine, aspartic acid, etc.) and are as identified in Peptidase Database MEROPS, available at the European Bioinformatics Institute with MEROPS identifier M10.004, see also, Rawlings, N.D., Barrett, A.J., Thomas, P.D., Huang, X., Bateman, A. & Finn, R.D. (2018) The MEROPS database of proteolytic enzymes, their substrates and inhibitors in 2017 and a comparison with peptidases in the PANTHER database. Nucleic Acids Res 46, D624-D632. “a” represents D- alanine.

In some embodiments, the cleavage site comprises an enzymatic cleavage site for a cathepsin. Cathepsins are a family of lysosomal hydrolases. According to their active site amino acid, cathepsins can be divided into three sub-groups: cysteine (B, C, H, F, K, L, O, S, V, W and X/Z), aspartate (D and E) and serine (G) cathepsins. The lysosome relies on these protein hydrolases and other enzymes to carry out intracellular degradation before recycling cellular constituents. In addition to its localization in the lysosome, cathepsins can be released from the cell and function to degrade components of the extracellular matrix. Overexpression of cathepsins have been observed in malignant tumors and has been found to be closely correlated with an array of cancers (invasive and metastatic). (Cao, Biomed. Pharmacother. 2019, 118, 109340; Cheng, Adv. Mater. Interfaces 2020, 7 (19), 1-9).

In some embodiments, the cleavage site comprises an enzymatic cleavage site for a Matrix metalloproteinase (MMP). MMPs is a large family, named for its need for metal ions as cofactors such as Ca²⁺ and Zn²⁺. It is the most prominent family of proteases associated with tumorigenesis. MMPs can degrade various protein components in the extracellular matrix and promote tumor invasion and metastasis by destroying the histological barrier during tumor invasion. Generally, MMPs are highly overexpressed in tumor tissues, and ischemia and hypoxia caused by tumor growth promote the expression of MMPs. (Cao, Biomed. Pharmacother. 2019, 118, 109340)

In some embodiments, the cleavage site comprises an enzymatic cleavage site for a Prostate-specific antigen (PSA). PSA is a type of serine protease and is commonly expressed in the epithelial cells of the prostate gland. Active PSA only exists around the prostate cells. Previous studies have found that the blood concentration of PSA will increase in the presence of prostate cancer or other prostate disorders. Therefore, PSA is used in targeted therapy for prostate cancer. (Zhang, Eur. J. Pharm. Biopharm. 2019, 137, 122-130.)

In some embodiments, the cleavage site comprises an enzymatic cleavage site for an elastase. High levels of elastase have been reported in primary tumors and metastasis, where it promotes oncogenic signaling and inhibits tumor suppressors. As a result, elevated neutrophil elastase levels correlate with poor prognosis in different types of solid tumors. The use of the peptide linker NPA as the substrate for neutrophil elastase was recently reported, and can be utilized as described herein. (Dias, Chemistry 2019; 25 (7): 1696-1700)

In some embodiments, the cleavage site comprises an enzymatic cleavage site for a plasmin. The plasmin system plays a key role in tumor invasion and metastasis by its matrix degrading activity and its involvement in tumor growth. In the body plasmin is predominantly present in its inactive pro-enzyme form plasminogen. Active plasmin is formed locally at or near the surface of tumor cells by urokinase-type plasminogen activator (uPA), produced by the cancer and/or stroma cells. (Franciscus, J. Med. Chem. 1999, 42 (25) 5277-5283).

In some embodiments, the water solubilizing group may be a group that is cleaved by one or more endogenous enzymes in a subject and/or biological sample such as one or more endogenous enzymes in circulation (e.g., blood circulation), extracellular space (e.g., a tumor extracellular space), and/or in a lysosome of a cell. In some embodiments, cleavage by one or more endogenous enzymes is at a cleavage site of a compound of the present invention, with optionally a portion of the cleavage site and the water solubilizing group cleaved from the compound. In some embodiments, the water solubilizing group comprises a group that aids in water solubilizing the compound, e.g., a group that may increase water solubility and/or modify (e.g., decrease) the clearance rate of the compound in vivo. Exemplary water solubilizing groups include, but are not limited to, a polyethylene glycol (PEG), glycoside, sulfonate, ammonium, carboxylate, betaine, phosphate, phosphonate, and/or peptide, optionally wherein the water solubilizing group comprises a carboxy-terminated group (e.g., a carboxy-terminated PEG group) and/or an amine terminated group. In some embodiments, the water solubilizing group comprises a glycoside such as, but is not limited to, glucoside, galactoside, glucuronide, and/or galacturonide. In some embodiments, the water solubilizing group comprises an amino acid and/or a peptide, optionally wherein the amino acid and/or peptide comprises one or more charged amino acids, including, but not limited to, arginine (R), lysine (K), histidine (H), aspartic acid (D), and/or glutamic acid (E) and/or one or more polar amino acids, including, but not limited to, serine(S), threonine (T), asparagine (N), and/or glutamine (Q). In some embodiments, the water solubilizing group comprises a polyethylene glycol (PEG) group, optionally wherein the PEG group comprises 1 to 24 PEG units, for example, 1, 2, 3, 4, 5, 6, 7, 8, 9, 10, 11, 12, 13, 14, 15, 16, 17, 18, 19, 20, 21, 22, 23 or 24 PEG units. In some embodiments, the PEG group has a structure of —(OCH₂CH₂)ₙ₁Y, where n1 is an integer of 1 to 24, for example, 1, 2, 3, 4, 5, 6, 7, 8, 9, 10, 11, 12, 13, 14, 15, 16, 17, 18, 19, 20, 21, 22, 23 or 24; and Y is —C(O)OH, —OCH₃, —OH, —PO₃H₂, —SO₃H, or —OCH₂C(O)OH.

The tetrapyrrole macrocycle of a compound of the present invention may include a porphyrin, chlorin, isobacteriochlorin, and bacteriochlorin, or a derivative thereof. Chlorins and bacteriochlorins and isobacteriochlorins may be regarded as derivatives of porphyrins. Exemplary tetrapyrroles include but are not limited to those described in U.S. Pat. Nos. 6,272,038; 6,451,942; 6,420,648; 6,559,374; 6,765,092; 6,407,330; 6,642,376; 6,946,552; 6,603,070; 6,849,730; 7,005,237; 6,916,982; 6,944,047; 7,884,280; 7,332,599; 7,148,361; 7,022,862; 6,924,375; 7,501,507; 7,323,561; 7,153,975; 7,317,108; 7,501,508; 7,378,520; 7,534,807; 7,919,770; 7,799,910; 7,582,751; 8,097,609; 8,187,824; 8,207,329; 7,633,007; 7,745,618; 7,994,312; 8,278,340; 9,303,165; and 9,365,722; and International Application Nos. PCT/US17/47266 and PCT/US17/63251. In embodiments, the tetrapyrrole macrocycle of the compound is a porphyrin. In some embodiments, the tetrapyrrole macrocycle has a structure of Formula Ia or Formula 1b:

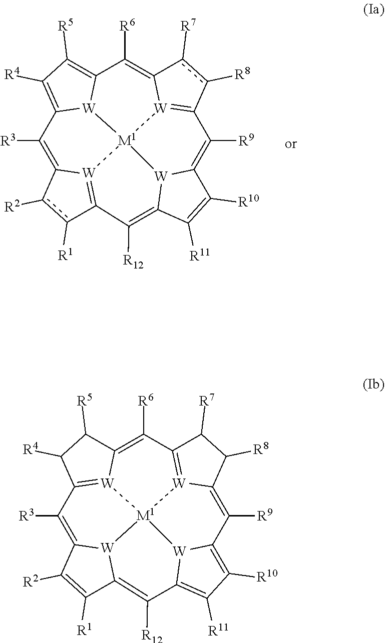

wherein:
[symbol] indicates a single bond or a double bond;
R¹, R², R³, R⁴, R⁵, R⁶, R⁷, R⁸, R⁹, R¹⁰, R¹¹, and R¹² are each independently selected from the group consisting of a hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, aWrylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, aminoacyloxy, hydrophilic groups, linking groups, bioconjugatable groups, surface attachment groups, targeting groups, the hydrogelator, the cleavage site, and/or the water solubilizing group, each of which may optionally be substituted;
or R¹ and R² together represent a fused aromatic or heteroaromatic ring system that is substituted or unsubstituted;
or R² and R³ together represent a fused aromatic or heteroaromatic ring system that is substituted or unsubstituted;
or R³ and R⁵ together represent a fused aromatic or heteroaromatic ring system that is substituted or unsubstituted;
or R⁴ and R⁵ together represent a fused aromatic or heteroaromatic ring system that is substituted or unsubstituted;
or R⁴ and R⁷ together represent a fused aromatic or heteroaromatic ring system that is substituted or unsubstituted;
or R⁷ and R⁸ together represent a fused aromatic or heteroaromatic ring system that is substituted or unsubstituted;
or R⁹ and R¹⁰ together represent a fused aromatic or heteroaromatic ring system that is substituted or unsubstituted; or
or R¹⁰ and R¹¹ together represent a fused aromatic or heteroaromatic ring system that is substituted or unsubstituted; and
M¹ is the radionuclide, and
W is N, O, S, Se, or CH; and
wherein at least one of R¹, R², R³, R⁴, R⁵, R⁶, R⁷, R⁸, R⁹, R¹⁰, R¹¹, and R¹² comprises the hydrogelator, cleavage site, and water solubilizing group or is substituted with the hydrogelator, cleavage site, and water solubilizing group.

In some embodiments, the tetrapyrrole macrocycle has a structure of Formula Ia and is a porphyrin having a structure of:

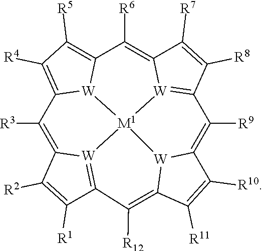

In some embodiments, the tetrapyrrole macrocycle has a structure of Formula Ia and is a chlorin having a structure of:

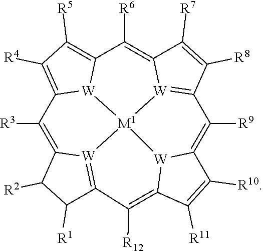

In some embodiments, the tetrapyrrole macrocycle has a structure of Formula Ia and is a bacteriochlorin having a structure of:

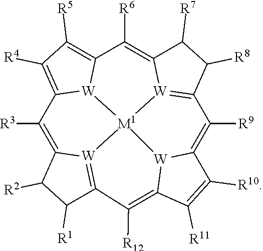

In some embodiments, the tetrapyrrole macrocycle has a structure of Formula Ib and is an isobacteriochlorin.

In some embodiments, the compound has a molecular weight of about 600 Daltons to about 1000 or 5000 Daltons, e.g., 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900 or 5000 Daltons. The molecular weight of the compound may be about 600 to about 5000 Daltons, 600 to about 4000 Dalton, 600 to about 3000 Daltons, 600 to about 2000 Daltons, or 600 to about 1000 Daltons. In some embodiments, the compound has a water solubility of at least 0.1 mg/mL, or at least 0.1 M. In some embodiments, the compound has a water solubility of at least 0.1 mg/mL, 0.15 mg/mL, 0.2 mg/mL, 0.25 mg/mL, 0.3 mg/mL, 0.35 mg/mL, 0.4 mg/mL, 0.45 mg/mL, 0.5 mg/mL, 0.55 mg/mL, 0.6 mg/mL, 0.65 mg/mL, 0.7 mg/mL, 0.75 mg/mL, 0.8 mg/mL, 0.85 mg/mL, 0.9 mg/mL, 0.95 mg/mL, or 1.0 mg/mL. In some embodiments, the compound has a water solubility of at least at least 0.1 M, at least 0.2 M, at least 0.3 M, at least 0.4 M, at least 0.5 M, at least 0.6 M, at least 0.7 M, at least 0.8 M, at least 0.9 M, or at least 1.0 M.

In some embodiments, the compound has a logarithm of partition coefficient (Log P) of less than 0, optionally wherein the compound has a Log P of about −0.25 to about −5. In some embodiments, the compound has a Log P of about −0.25 to about −4, about −0.25 to about −3, about −0.25 to about −2, about 0 to about −4, about 0 to about −3.5, about 0 to about −3, about 0 to about-2.5, or about 0 to about −2.

In some embodiments, the compound has a 1-octanol to aqueous composition partitioning ratio in a range of about 1:1.5 or 1:2 to about 1:100 or 1:100,000 (1-octanol: aqueous composition), wherein the aqueous composition is deionized water or phosphate buffered saline at pH 7.4, optionally wherein the PBS is about 0.1 M. In some embodiments, the compound has a 1-octanol to aqueous composition partitioning ratio in a range of about 1:1.5 to about 1:100,000, about 1:1.5 to about 1:75,000, 1:1.5 to about 1:50,000, about 1:1.5 to about 1:25,000, about 1:1.5 to about 1:10,000, about 1:1.5 to about 1:7,500, about 1:1.5 to about 1:5,000, about 1:1.5 to about 1:2,500, about 1:1.5 to about 1:1,000, about 1:1.5 to about 1:750, about 1:1.5 to about 1:500, about 1:1.5 to about 1:250, about 1:1.5 to about 1:100, about 1:2 to about 1:100,000, about 1:2 to about 1:75,000, 1:2 to about 1:50,000, about 1:2 to about 1:25,000, about 1:2 to about 1:10,000, about 1:2 to about 1:7,500, about 1:2 to about 1:5,000, about 1:2 to about 1:2,500, about 1:2 to about 1:1,000, about 1:2 to about 1:750, about 1:2 to about 1:500, about 1:2 to about 1:250, or about 1:2 to about 1:100.

In some embodiments, the compound comprises two or more (e.g., 3, 4, 5, 6, or more) hydrogelators, cleavage sites, and/or water solubilizing groups.

In some embodiments, a hydrogelator, cleavage site, and/or water solubilizing group is attached at the perimeter of the tetrapyrrole macrocycle. In some embodiments, a hydrogelator, cleavage site, and/or water solubilizing group is attached at the perimeter of a compound having a structure of Formula Ia or Formula Ib. In some embodiments, a hydrogelator, cleavage site, and/or water solubilizing group is attached at the perimeter of a porphyrin, chlorin, bacteriochlorin, or isobacteriochlorin.

In some embodiments, the tetrapyrrole macrocycle comprises a swallowtail group and the hydrogelator, cleavage site, and/or water solubilizing group is bound (directly or indirectly) to an atom of the swallowtail group. In some embodiments, a tetrapyrrole molecule has a structure of Formula Ia or Formula Ib and a swallowtail group is attached at one of R¹, R², R³, R⁴, R⁵, R⁶, R⁷, R⁸, R⁹, R¹⁰, R¹¹, and R¹², optionally at R³, R⁶, R⁹, and/or R¹². In some embodiments, the swallowtail group has a structure of —CH((CH₂)ₙ₂(C(O)O)ₘ₂(CH₂)ₚ₂Y)₂, wherein: n2 is an integer of 1 to 20; m2 is 0 or 1; p2 is 0 or an integer of 1 to 10; and Y is absent, —OCH₂—, —C(O)— or —C(O)O—. In some embodiments, the swallowtail group has a structure of —CH((CH₂)ₙ₃(OCH₂CH₂)ₘ₃X(CH₂)ₚ₃Y)₂, wherein: n3 is an integer of 1 to 20; m3 is 0 or 1 to 24; X is oxygen or absent; p3 is an integer of 1 to 10; and Y is absent, —C(O)O—, —OCH₂—, —O—, —C(O)—, —PO₃H—, —SO₃—, or —OCH₂C(O)O—. In some embodiments, the swallowtail group has a structure of —CH((CH₂)ₙ₄(C(O)NH)(CH₂)ₘ₄(OCH₂CH₂)ₚ₄Y)₂, wherein: n4 is an integer of 1 to 20; m4 is 1 to 20; p4 is an integer of 0, or 1 to 24; and Y is absent, —C(O)O—, —OCH₂—, —O—, —C(O)—, —PO₃H—, —SO₃—, or —OCH₂C(O)O—.

In some embodiments, a compound of the present invention comprises a tetrapyrrole macrocycle having a structure of Formula Ia or Formula Ib with a swallowtail group attached (directly or indirectly) at the perimeter of the tetrapyrrole macrocycle (e.g., at one of R¹, R², R³, R⁴, R⁵, R⁶, R⁷, R⁸, R⁹, R¹⁰, R¹¹, and R¹²) and a hydrogelator, cleavage site, and/or water solubilizing group is bound (directly or indirectly) to an atom of the swallowtail group (e.g., at an end (e.g., terminus) of the swallowtail group). In some embodiments, the tetrapyrrole macrocycle comprises a porphyrin, a chlorin, a bacteriochlorin, or an isobacteriochlorin with a swallowtail group attached (directly or indirectly) at the perimeter of the tetrapyrrole macrocycle (e.g., at one of R¹, R², R³, R⁴, R⁵, R⁶, R⁷, R⁸, R⁹, R¹⁰, R¹¹, and R¹²) and a hydrogelator, cleavage site, and/or water solubilizing group is bound (directly or indirectly) to an atom of the swallowtail group (e.g., at an end (e.g., terminus) of the swallowtail group). In some embodiments, a hydrogelator, cleavage site, and/or water solubilizing group is attached (e.g., indirectly) at the 5-position and/or 15-position of a tetrapyrrole macrocycle having a structure of Formula Ia or Formula Ib, optionally via a swallowtail group that is attached at the 5-position and/or 15-position of the tetrapyrrole macrocycle. In some embodiments, a compound of the present invention comprises a first moiety that includes a first hydrogelator, a first cleavage site, and a first water solubilizing group and a second moiety that includes a second hydrogelator, a second cleavage site, and a second water solubilizing group, and the first moiety is attached at the 5-position of a tetrapyrrole macrocycle having a structure of Formula Ia or Formula Ib and the second moiety is attached at the 15-position of the tetrapyrrole macrocycle, optionally wherein the first moiety and the second moiety are the same or different. In some embodiments, a hydrogelator, cleavage site, and/or water solubilizing group is attached at a beta-position or a meso-position of the tetrapyrrole macrocycle, optionally having a structure of Formula Ia or Formula Ib. In some embodiments, the compound further comprises a first linker, wherein the first linker is attached to the tetrapyrrole macrocycle, optionally having a structure of Formula Ia or Formula Ib, and to the hydrogelator. In some embodiments, a hydrogelator, cleavage site, and/or water solubilizing group is attached at the perimeter of a tetrapyrrole macrocycle. In some embodiments, a hydrogelator, cleavage site, and/or water solubilizing group is attached at the perimeter of a tetrapyrrole macrocycle having a structure Formula Ia or Formula Ib. In some embodiments, a hydrogelator, cleavage site, and/or water solubilizing group is attached at the perimeter of a porphyrin, chlorin, bacteriochlorin or isobacteriochlorin. In some embodiments, the tetrapyrrole macrocycle, optionally having a structure of Formula Ia or Formula Ib, comprises an aryl moiety and the hydrogelator, cleavage site, and/or water solubilizing group is bound (directly or indirectly) to an atom of the aryl moiety. The aryl moiety may comprise a phenyl group, optionally wherein the phenyl group is substituted with the hydrogelator, cleavage site, and/or water solubilizing group at an ortho position relative to attachment of the phenyl group to the tetrapyrrole macrocycle. The aryl moiety may comprise a phenyl group, optionally wherein the phenyl group is optionally disubstituted with the hydrogelator, cleavage site, and/or water solubilizing group. The aryl moiety may comprise a phenyl group, optionally wherein the phenyl group is substituted two hydrogelators, cleavage sites, and/or water solubilizing groups at an ortho position relative to attachment of the phenyl group to the tetrapyrrole macrocycle.

In some embodiments, a hydrogelator, cleavage site, and/or water solubilizing group is/are attached at the 2-position and/or 6-position of the aryl moiety. In some embodiments, the compound comprises a first moiety that includes a first hydrogelator, a first cleavage site, and a first water solubilizing group and a second moiety that includes a second hydrogelator, a second cleavage site, and a second water solubilizing group, and the first moiety is attached at the 2-position of the aryl moiety and the second moiety is attached at the 4-position, 5-position, or 6-position of the aryl moiety, or the first moiety is attached at the 3-position or the 4-position of the aryl moiety and the second moiety is attached at the 6-position of the aryl moiety. In some embodiments, the first moiety and the second moiety are the same. In some embodiments, the first moiety and the second moiety are different. In some embodiments, a hydrogelator, cleavage site, and/or water solubilizing group is attached at the 3-position and/or 5-position of the aryl moiety. In some embodiments, the compound comprises a first moiety that includes a first hydrogelator, a first cleavage site, and a first water solubilizing group and a second moiety that includes a second hydrogelator, a second cleavage site, and a second water solubilizing group, and the first moiety is attached at the 3-position of the aryl moiety and the second moiety is attached at the 5-position of the aryl moiety, optionally wherein the first moiety and the second moiety are the same or different.

In some embodiments, the tetrapyrrole macrocycle has a structure of Formula Ia or Formula Ib (e.g., a porphyrin, bacteriochlorin, chlorin, or isobacteriochlorin) and a hydrogelator, cleavage site, and/or water solubilizing group is attached at the 5-position and/or 15-position of the tetrapyrrole macrocycle. In some embodiments, the compound comprises a first moiety that includes a first hydrogelator, a first cleavage site, and a first water solubilizing group and a second moiety that includes a second hydrogelator, a second cleavage site, and a second water solubilizing group, and the first moiety is attached at the 5-position of a tetrapyrrole macrocycle having a structure of Formula Ia or Formula Ib and the second moiety is attached at the 15-position of the tetrapyrrole macrocycle, optionally wherein the first moiety and the second moiety are the same or different. In some embodiments, a hydrogelator, cleavage site, and/or water solubilizing group is attached at a beta-position or a meso-position of a tetrapyrrole macrocycle optionally having a structure of Formula Ia or Formula Ib. In some embodiments, the compound further comprises a first linker, wherein the first linker is attached to the tetrapyrrole macrocycle, optionally having a structure of Formula Ia or Formula Ib, and to the hydrogelator.

The compound may further comprise a targeting agent (e.g., 1, 2, 3, 4 or more targeting agent(s)), optionally wherein the one or more targeting agent(s) is a cancer target agent. In some embodiments, the compound comprises at least two targeting agents. In some embodiments, the compound further comprises a second linker, wherein the second linker is attached to the tetrapyrrole macrocycle and to the targeting agent. Exemplary targeting agents include, but are not limited to, antibodies, peptides, and/or receptors. In some embodiments, the targeting agent is an antibody or fragment thereof, optionally wherein the targeting agent is a monoclonal antibody (mAb) or fragment thereof. Exemplary antibody fragments include, but are not limited to, camelid-derived heavy chain antibodies (HCAbs) and the variable domain of the heavy chain antibodies (VHH), also termed nanobodies. The latter are small (about 15 kDa) and may afford better tumor penetration than the larger full antibodies. In some embodiments, a targeting agent (e.g., antibody) may not recognize every tumor cell type, and instead may recognize only a subset (e.g., a subset that is present in every tumor and every metastasis). In some embodiments, the target for a targeting agent (e.g., an antibody) is a glucosidase (e.g., a β-glucosidase) and/or a glucuronidase (e.g., a β-glucuronidase). Further exemplary targeting agents include, but are not limited to, those described in U.S. Pat. Nos. 7,807,136 and 7,615,221. Binding of the targeting agent and target may allow for the target to maintain its activity. In some embodiments, an agent to which a cancer cell targeting agent binds (e.g., a receptor) is expressed on cancer cells at a concentration that is greater than non-cancerous cells such as, for example, at a concentration that is about 2, 3, 4, 5, 6, 7, 8, 9, or 10-fold higher or more. In some embodiments, the targeting agent is any agent or compound that directs the compound to a given target cellular destination such as a cancer cell and/or tumor extracellular space. In some embodiments, a targeting agent binds to and/or targets a receptor on a cell surface such that a compound of the present invention or a portion thereof is bound to the cell surface and/or remains in extracellular space. In some embodiments, a targeting agent (e.g., antibody and/or nanobody) may be substituted with one or more substituent(s), linker(s), and/or water solubilizing group(s), optionally to modify the water solubility and/or clearance time of the compound. In some embodiments, a targeting agent (e.g., antibody and/or nanobody) may be substituted with one or more PEG group(s), optionally to modify the water solubility and/or clearance time of the compound.

In some embodiments, a linker (e.g., a first linker and/or a second linker) may comprise a hydrocarbon (e.g., an alkyl group), swallowtail group, aryl (e.g., phenol), heterocyclic ring (e.g., a 1,3,5-triazine, 1,2,3-triazole, etc.), amino acid residue (e.g., a D-amino acid residue and/or an L-amino acid residue), peptide, ether, ketone, ester, amide, branched polymer (e.g., a poly(amidoamine) dendrimer), and/or linear polymer (e.g., polyethylene glycol (PEG)), each of which may be unsubstituted or substituted. Further exemplary linkers include, but are not limited to, those described in Ertl et al., The most common linkers in bioactive molecules and their bioisosteric replacement network, Bioorg. Med. Chem. 81 (2023) 117194, which is incorporated herein by reference in its entirety. In some embodiments, a compound of the present invention comprises a first linker and a second linker that are the same. In some embodiments, a compound of the present invention comprises a first linker and a second linker that are different. Linkers may be selected, for example, based on their orientation in space, water solubility, length, biocompatibility, and/or degradation profile/stability.

According to some embodiments provided are compositions such as, e.g., pharmaceutical compositions. A pharmaceutical composition of the present invention may comprise a therapeutically effective amount of a compound of the present invention (e.g., a first agent, a second agent, and/or a third agent as described herein) in a pharmaceutically acceptable carrier. Pharmaceutical carriers suitable for administration of a compound of the present invention include any such carriers known to those skilled in the art to be suitable for the particular mode of administration. In some embodiments, a pharmaceutical composition of the present invention is a composition as described in U.S. Pat. Nos. 7,807,136 and 7,615,221 with the active ingredient replaced with a compound of the present invention as the active ingredient.

In some embodiments, a compound of the present invention (i.e., active ingredient) may be formulated as the sole pharmaceutically active ingredient in the composition or may be combined with other active ingredients.

A composition of the present invention may comprise one or more compounds of the present invention. In some embodiments, the compounds may be formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In some embodiments, the compounds described herein are formulated into pharmaceutical compositions using techniques and procedures well known in the art (see, e.g., Ansel, Introduction to Pharmaceutical Dosage Forms, Fourth Edition 1985, 126).

In the compositions, effective concentrations of one or more compounds or pharmaceutically acceptable derivatives thereof may be (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation. The concentrations of the compounds in the compositions may be effective for delivery of an amount, upon administration, that treats cancer and/or one or more of the symptoms in a subject and/or kills one or more cancer cells in a subject.

In some embodiments, the compositions are formulated for single dosage administration. To formulate a composition, the weight fraction of a compound of the present invention is dissolved, suspended, dispersed or otherwise mixed in a selected carrier at an effective concentration such that the treated condition is relieved, prevented, or one or more symptoms may be ameliorated.

The active compound may be included in the pharmaceutically acceptable carrier in an amount sufficient to exert a therapeutically useful effect in the absence of undesirable side effects on the subject treated. The therapeutically effective concentration may be determined empirically by testing the compounds in in vitro and/or in vivo systems described herein and in U.S. Pat. No. 5,952,366 to Pandey et al. (1999) and then extrapolated therefrom for dosages for humans.

The concentration of an active compound in the pharmaceutical composition may depend on absorption, inactivation and excretion rates of the active compound, the physicochemical characteristics of the compound, the dosage schedule, and/or the amount administered as well as other factors known to those of skill in the art. For example, the amount that is delivered may be sufficient to kill one or more cancer cells as described herein.

In some embodiments, a therapeutically effective dosage should produce a serum concentration of the active ingredient of from about 0.1 ng/ml to about 50-100 μg/ml. In one embodiment, a therapeutically effective dosage is from about 0.001, 0.01 or 0.1 to about 10, 100 or 1000 mg of active compound per kilogram of body weight per day. Pharmaceutical dosage unit forms may be prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo and/or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN™, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration may be sufficient for ameliorating the symptoms of the disease, disorder or condition treated and may be empirically determined.

The pharmaceutical compositions may be provided for administration to humans and/or animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Liquid pharmaceutically administrable compositions may, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrin derivatives, sorbitan monolaurate, triethanolamine sodium acetate, triethanolamine oleate, and other such agents.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of about 0.005% to about 100% with the balance made up from non-toxic carrier may be prepared. Methods for preparation of these compositions are known to those skilled in the art. The contemplated compositions may contain about 0.001%-100% active ingredient, in one embodiment about 0.1-95%, in another embodiment about 75-85%.

In some embodiments, a composition of the present invention may be suitable for oral administration. Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, in one embodiment, capsules or tablets. The tablets, pills, capsules, troches and the like may contain one or more of the following ingredients, or compounds of a similar nature: a binder; a lubricant; a diluent; a glidant; a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, gellan gum, sodium carboxymethylcellulose, polyethylene glycol 4000 and cellulose acetate phthalate.

The compound, or pharmaceutically acceptable derivative thereof, may be provided in a composition that protects it from the acidic environment of the stomach. For example, the composition may be formulated in an enteric coating that maintains its integrity in the stomach and releases the active compound in the intestine. The composition may also be formulated in combination with an antacid or other such ingredient. When the dosage unit form is a capsule, it may contain, in addition to material of the above type, a liquid carrier such as a fatty oil. In addition, dosage unit forms may contain various other materials which modify the physical form of the dosage unit, for example, coatings of sugar and other enteric agents. The compounds may be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials may also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In some embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms. Solvents include glycerin, sorbitol, ethyl alcohol and syrup. Examples of preservatives include glycerin, methyl and propylparaben, benzoic acid, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, xanthan gum, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation. For a solid dosage form, the solution or suspension, in for example propylene carbonate, vegetable oils or triglycerides, is in one embodiment encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(loweralkyl) acetals of loweralkyl aldehydes such as acetaldehyde diethyl acetal.

Parenteral administration, in one embodiment characterized by injection, either subcutaneously, intramuscularly or intravenously is also contemplated herein. Injectables may be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, xanthan gum, hydroxypropyl methylcellulose and polyvinylpyrrolidone. Emulsifying agents include Polysorbate 80 (TWEEN™ 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound may be adjusted so that an injection provides an effective amount to produce the desired pharmacological effect. The exact dose depends on the age, weight and condition of the subject or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.01% or 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

In some embodiments, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The present invention finds use in both veterinary and medical applications. Subjects suitable to be treated with a method of the present invention include, but are not limited to, mammalian subjects. Mammals of the present invention include, but are not limited to, canines, felines, bovines, caprines, equines, ovines, porcines, rodents (e.g. rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Mammalian (e.g., human) subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult. In some embodiments, the subject has or is believed to have cancer, optionally wherein the subject has metastatic cancer.

A method of the present invention may also be carried out on animal subjects, particularly mammalian subjects such as mice, rats, dogs, cats, livestock and horses for veterinary purposes, and/or for drug screening and drug development purposes.

In some embodiments, the subject is “in need of” or “in need thereof” of a method of the present invention, for example, the subject has findings typically associated with cancer and/or a tumor, is suspected to have cancer and/or a tumor, and/or the subject has cancer and/or a tumor.

According to some embodiments of the present invention provided is a method of diagnosing a disease or disorder in a subject, the method comprising administering a compound of the present invention to the subject, thereby diagnosing the disease or disorder in the subject.

In some embodiments, a method of treating a subject in need thereof is provided, the method comprising administering a compound of the present invention to the subject, thereby treating the subject. According to some embodiments of the present invention provided is a method of treating a subject (e.g., a subject having a solid tumor) and/or reducing the size of a solid tumor in a subject, the method comprising administering a compound of the present invention to the subject, thereby treating the subject and/or reducing the size of the solid tumor in the subject.

In some embodiments, a compound of the present invention is used in a method of treating, detecting and/or diagnosing a disease or disorder, e.g., cancer, optionally in a subject. Advantageously, the present compounds are designed for use in methods where the compound targets a tumor tissue, and can undergo enzymatic processing (e.g. cleavage) at and/or around the tumor site, transforming the compound from a water soluble, monomeric state to a highly associated form.

In some embodiments, a compound of the invention is administered to a subject, wherein the compound is delivered to a tumor and immobilizes the radionuclide in and/or around the tumor. In some embodiments, a subject may be treated with a single radiolabeled compound. Administration of the compound may be chronically or intermittently over about 1, 2, 3, 4, 5, 6, 7, or more days to about 1, 2, 3, 4, or more weeks. In some embodiments, a compound may be administered in a manner to allow the compound and/or therapeutic agent and/or radionuclide to accumulate in and/or around a tumor mass. The compound may localize in an area where both an enzyme that can cleave a water solubilizing group of the compound and the target of the compound are present. In some embodiments, the compound is administered intravenously.

In some embodiments, a method of the present invention may comprise detecting the compound and/or radionuclide in the subject. The method can comprise, for example, administering a compound of the present invention that associates and/or immobilized in a cell, or tissue, and/or tumor site; and detecting the compound or a portion thereof, thereby detecting the cell, tissue, and/or agent. In some embodiments, a method of detecting a cell, tissue, and/or tumor in a subject is provided, the method comprising: administering to the subject a compound of the present invention, optionally wherein the compound associates with the cell, tissue, and/or tumor or otherwise aggregates or immobilizes at the cell, tissue and/or tumor site; and detecting the compound or a portion thereof within the subject, thereby detecting the cell, tissue, and/or tumor. In some embodiments, the subject has or is suspected to have cancer.

In some embodiments, a method of the present invention further comprises imaging the subject, optionally wherein the imaging is Magnetic Resonance Imaging (MRI), positron emission tomography (PET), and/or Computed Tomography (CT) (e.g., single-photon emission computed tomography (SPECT)).

In some embodiments, a method of aggregating and/or immobilizing a radionuclide in a subject is provided, the method comprising administering a compound of the present invention to the subject, thereby aggregating and/or immobilizing the radionuclide in the subject. In some embodiments, a cleavage agent (e.g., an enzyme) present in the subject cleaves the compound at the cleavage site, optionally wherein the cleavage agent is a phosphatase, cathepsin, matrix metalloproteinase, serine protease, elastase, urokinase, or urokinase-type plasminogen activator. In some embodiments, the cleavage agent is overexpressed in a region of the subject comprising a tumor. As described herein, the compounds can be designed to comprise a cleavage site responsive to a cleavage agent overexpressed in the region. In some embodiments, the compound is responsive to cleavage at the cleavage site and the water solubility of the compound decreases. The method of aggregating and/or immobilizing a radionuclide in a subject can comprise two or more compounds of the present invention that self-assemble (e.g., aggregate) in the subject, optionally wherein the two or more compounds self-assemble at and/or around a tumor in the subject.

The present invention is explained in greater detail in the following non-limiting examples.

### EXAMPLES

### Example 1

Enzyme-instructed self-assembly (EISA) is a process that integrates enzymatic reaction and self-assembly. Porphyrin compounds have stable structures, high molecular symmetry, and strong absorption of visible light and photosensitivity properties. Functional groups can be substituted on the porphyrin cycle at meso- or β-position to obtain various porphyrin derivatives. Moreover, the central cavity of porphyrin compounds can be chelated with various metals.

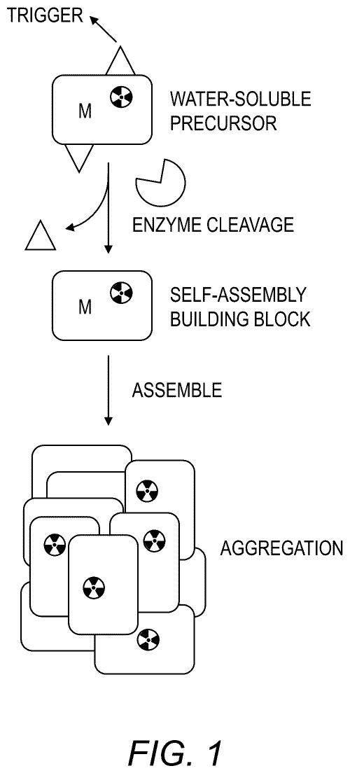

A new strategy in radiopharmaceutical applications was explored where water-soluble precursors bearing radionuclides can assemble into supramolecular aggregation upon triggering by a tumor-related enzyme and immobilize at tumor area (FIG. 1).

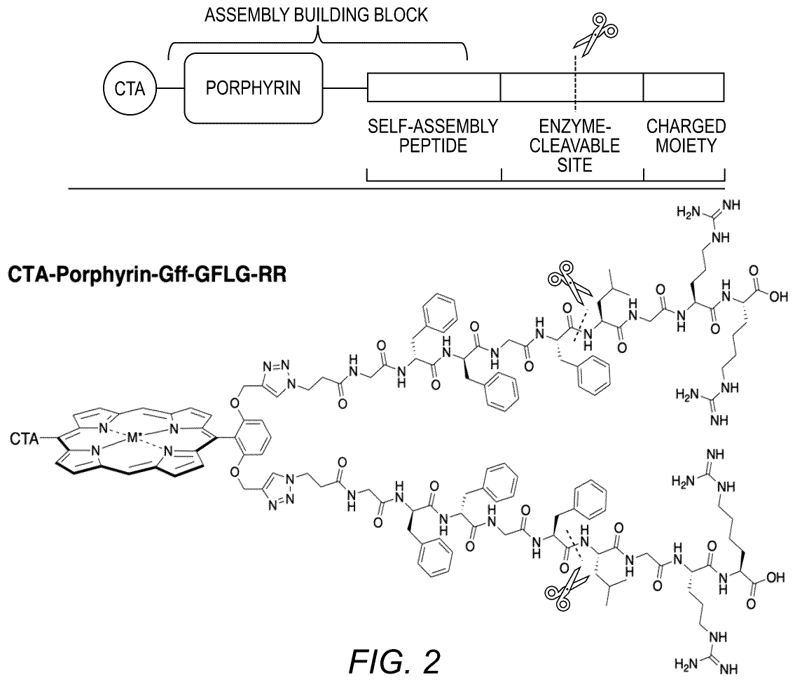

Here, a general design for targeting and immobilization of a radionuclide in a tumor has been developed (FIG. 2). The exemplary design includes (1) an optional cancer targeting agent, (2) a porphyrin bearing a radionuclide, (3) a peptide for self-assembly, also known as a hydrogelator, (4) an enzymatically cleavable group, and (5) a water solubilizing group that is removed upon enzymatic cleavage. In some embodiments, the cleavable group and the water-solubilizing water solubilizing group are one and the same, namely phosphate, which is removed by a phosphatase. In addition to phosphatase, other tumor-related enzymes could be incorporated into the design of the porphyrin-peptide conjugates as the EISA substrate.

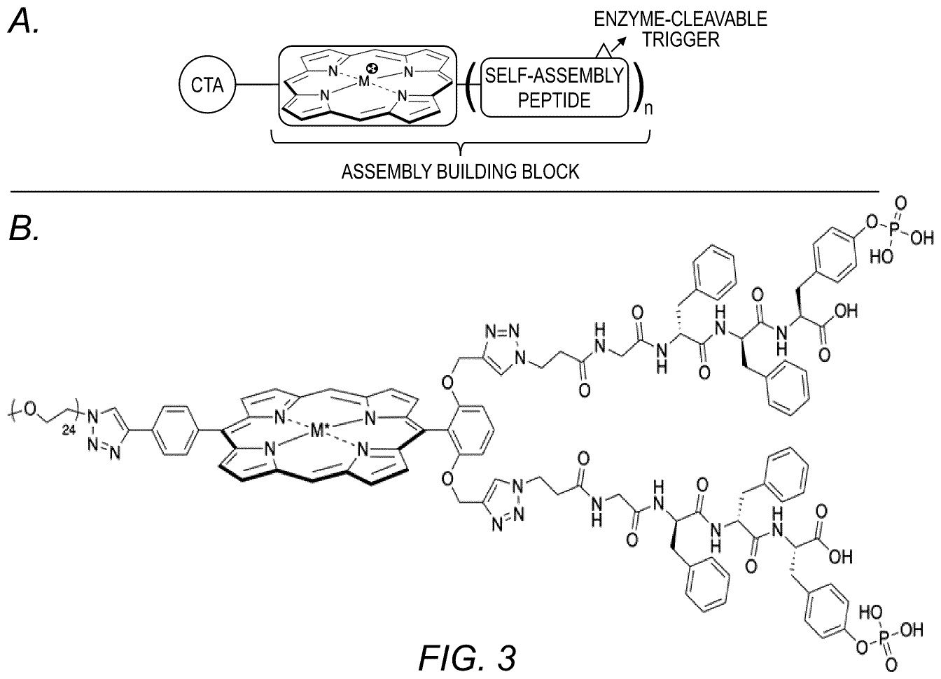

A porphyrin-peptide structure was designed (FIG. 3, panel A). The molecule bears a porphyrin hub carrying a radionuclide, multiple self-assembly peptide precursors, and a cancer targeting agent. Upon the treatment of the enzyme overexpressed in tumor region, the precursor will be triggered and allow the molecule to assemble. The structure of the model molecule is shown in FIG. 3, panel B. The peptide precursor GffY(p) is a phosphorylated tetrapeptide. Upon the treatment of ALP, the peptide will be dephosphorylated and transform into a self-assembly peptide GffY. The peptide precursors are attached to a porphyrin, which serves as a radionuclide carrier and provides π-π interaction in assemblies. The porphyrin also bears a clickable handle which allows the installation of cancer targeting agent (CTA). For the study, a PEG linker is attached instead of the CTA to provide water solubility. The target molecule is expected to bear the following features: (1) targeting tumor tissue; (2) delivering radionuclides; (3) aggregation and immobilization upon the treatment of the enzyme specially overexpressed at the tumor.

### Synthesis of the Pegylated Porphyrin-Peptide Conjugates (5)

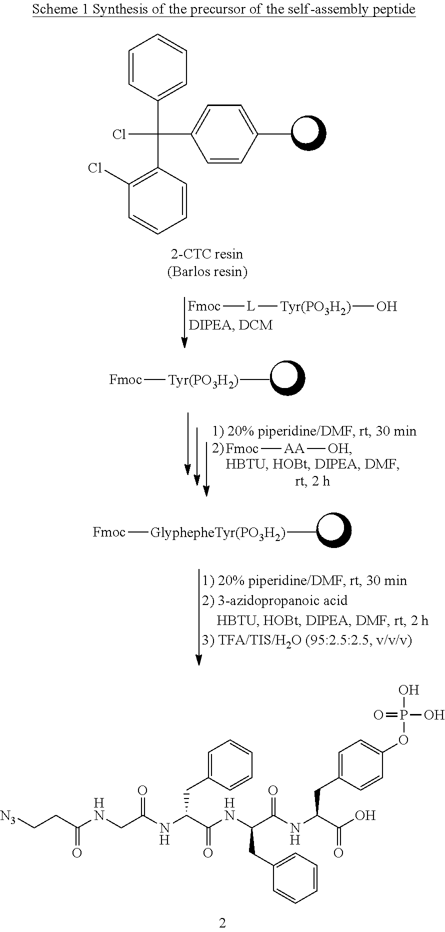

The precursor of the self-assembly peptide with an azide group on the N-terminus, 2, was synthesized following standard solid phase peptide synthesis protocol (Scheme 1). The tetrapeptide bears two D-phenylalanine residues, one phosphate-protected L-tyrosine residue, and one glycine as a spacer. The D-amino acids provide resistance toward protease digestion. Upon the treatment of the phosphatase, the phosphate group will be cleaved and allow the peptide to form hydrogel at a certain concentration. The peptide was synthesized in 2-CTC resin, with HBTU/HOBt as coupling reagents and DIPEA as the base. Fmoc-protected amino acids and 3-aizdoproopanoic acid were loaded in order, followed by the cleavage with TFA/TIS/H₂O (v/v/v, 95:2.5:2.5). The crude was purified with reverse phase preparative HPLC.

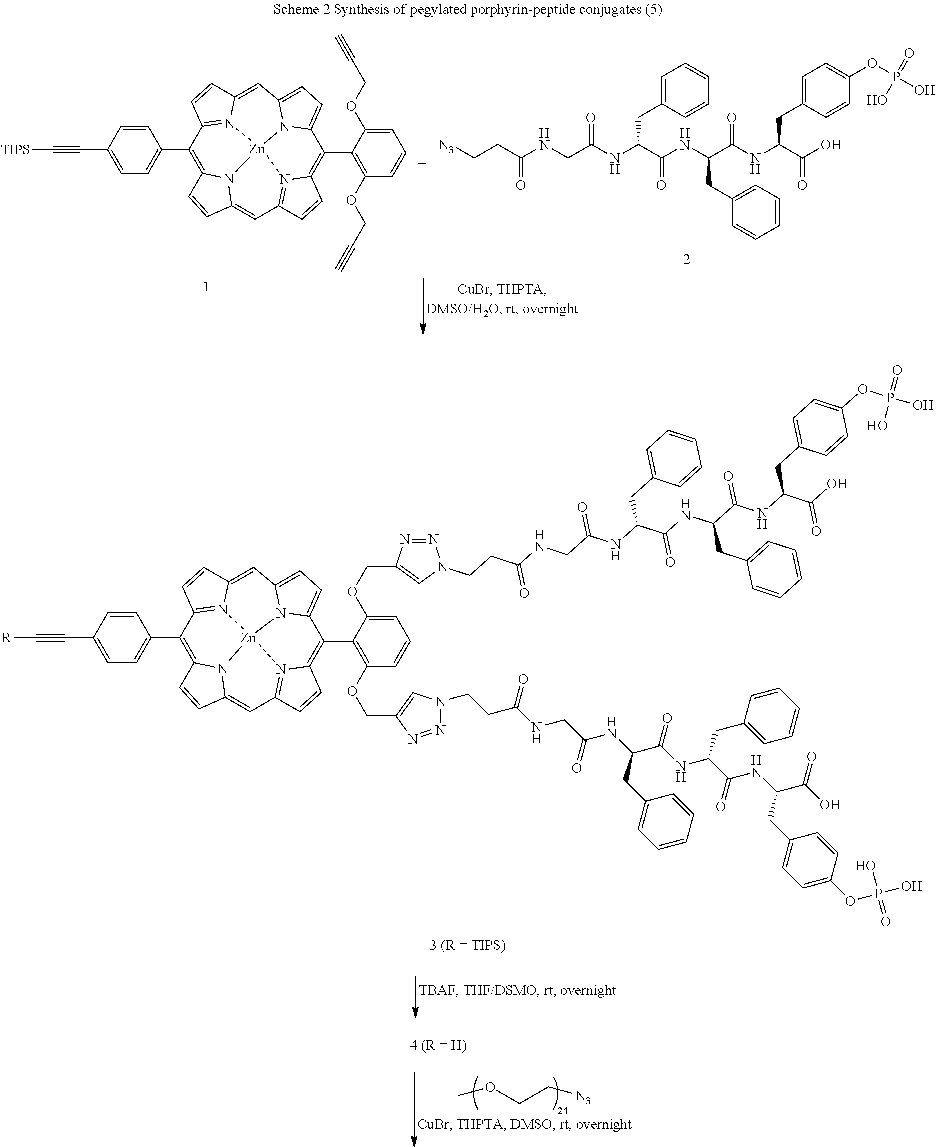

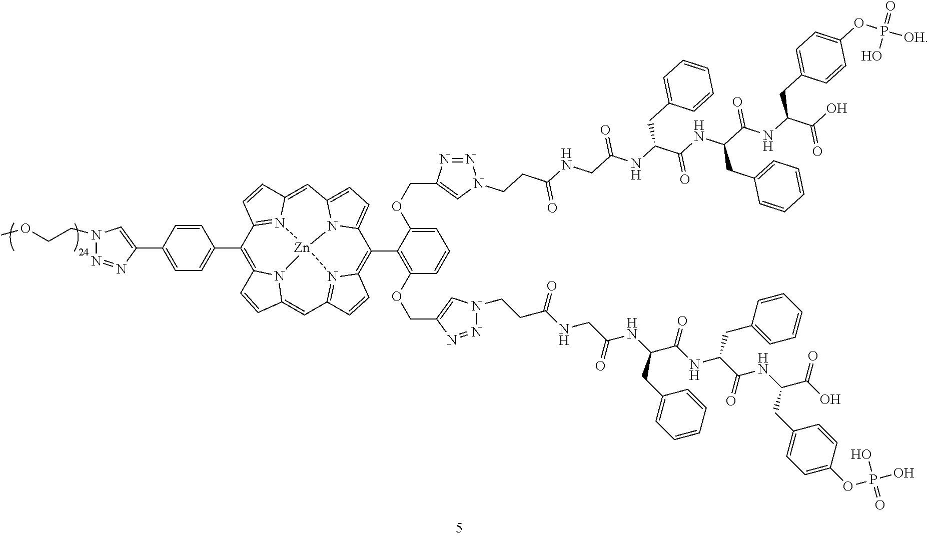

The peptides were attached to 1 via copper-catalyzed click chemistry. The reaction mixture was simply washed to remove the salts and the crude of 3 was used in the next step without further purification. The triisopropylsilyl group was removed with tetra-n-butylammonium fluoride to give compound 4. For better water solubility, a PEG₂₄ linker was tethered to 4 to yield 5 via copper-catalyzed click chemistry (Scheme 2).

Enzymatic Study with Alkaline Phosphatase (ALP)

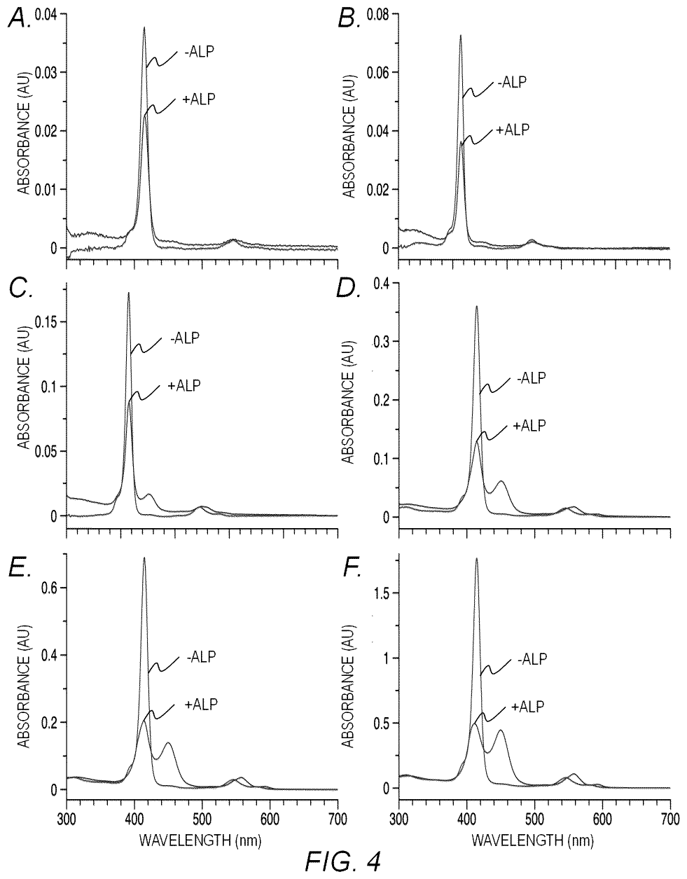

The stock solution of 5 in DMSO was diluted with PBS to prepare sample solutions with different concentrations (0.10 μM, 0.20 μM, 0.50 μM, 1.0 μM, 2.0 μM, and 5.0 μM). Each sample was treated with 2 U/mL of alkaline phosphatase (ALP) and incubated overnight at 37° C. The absorption spectra across the range 300-700 nm, where the porphyrin absorbs strongly, were measured before and after the enzyme treatment for each sample (FIG. 4, panels A-F). For samples with concentration lower than 0.50 μM (FIG. 4, panels A, B), the intensity of the Soret band of 5 at 420 nm decreased after enzyme treatment, yet precipitation or other signs of aggregation were not observed. When the concentration of the sample was equal to or higher than 0.50 μM (FIG. 4, panels C-F), the intensity of the Soret peak at 420 nm decreased and a new peak appeared at 455 nm after the enzyme treatment. The new peak at 455 nm indicates the aggregation of the sample upon the treatment of ALP. The Q band (at ˜550 nm) also underwent a bathochromic shift and broadening, albeit of lesser magnitude than that of the higher-energy Soret band. Compound 5′, which lacks the two phosphoester moieties, is prone to aggregation in PBS, consistent with the observed results for ALP treatment of 5. The change in the color of the sample was also observed. After the overnight incubation, the color of the sample changed from pink to orange.

Samples of 5 (10 μM in PBS) with or without ALP treatment were centrifuged with Amicon centrifugation devices (molecular cutoff 50 kDa). After centrifuging for 10 min at 10000 rpm, the sample without ALP treatment passed through the 50 kDa membrane and was collected in the bottom tube, while the sample after enzyme treatment didn't pass through the membrane and was concentrated in the top tube. No color was observed for the solution collected in the bottom tube for the sample that underwent enzyme treatment. Solutions collected in the bottom or top tube for the sample that underwent enzyme treatment were analyzed with absorption spectroscopy (FIG. 5). The solution from the bottom showed no absorption while the solution from the top (diluted by 20-fold) showed the absorption of the aggregated porphyrin-peptide conjugate.

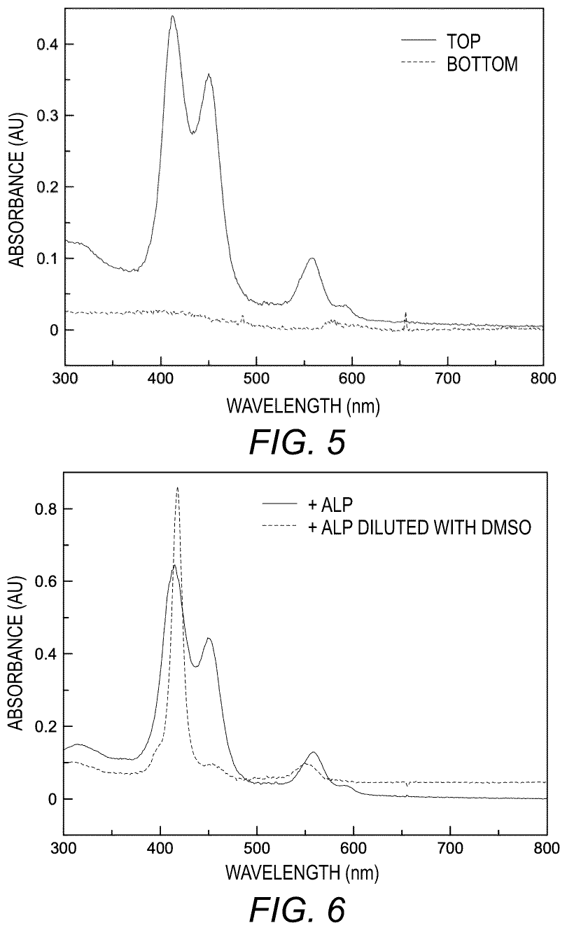

To confirm that the peak appeared after enzyme treatment was caused by aggregation, DMSO was added to the sample of 5 (5.0 μM in PBS) after enzyme treatment to disaggregate the sample. The absorption spectrum of the sample after adding DMSO is shown in FIG. 6. After adding DMSO to dilute the solution in half, the peak at 455 nm almost disappeared and the intensity of the peak at 420 nm increased. Color changes of the solution were observed during the treatment, with the solution appearing pink initially (indicating a monomeric state), then yellow after treatment with ALP (indicating aggregation), and finally returning to pink after dilution with DMSO, albeit a lighter pink than the initial solution, due to dilution (indicating disaggregation). The result further confirmed that compound 5 aggregates in aqueous solution after the enzyme treatment. Absorption spectral changes can be observed at 50-fold lower concentrations as shown in FIG. 4.

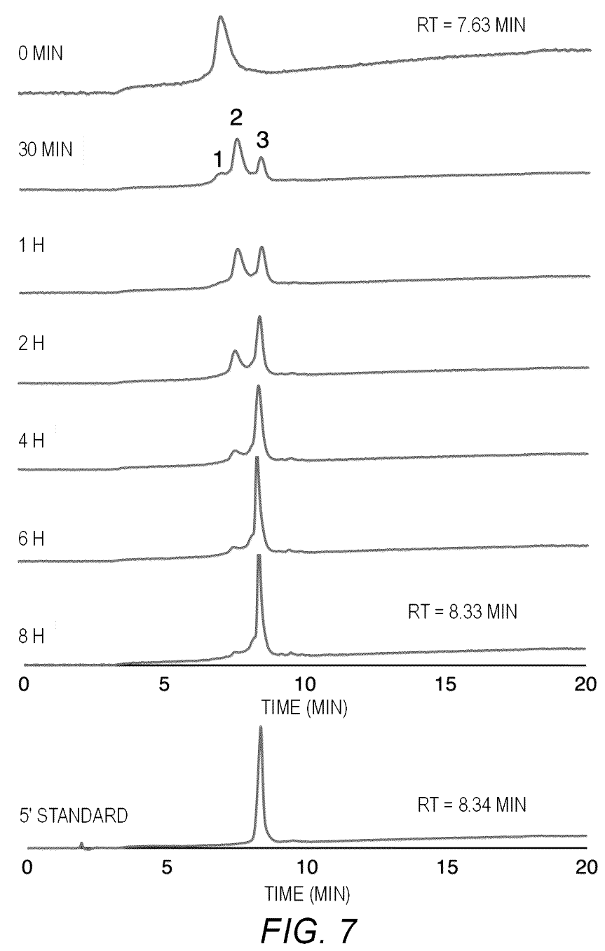

The enzymatic cleavage was monitored with reverse-phase high performance liquid chromatography. A solution of 200 μM 5 in PBS was treated with 2 U/mL ALP and incubated at 37° C. A small part of sample was taken from the reaction at different time points (30 min, 1 h, 2 h, 4 h, 6 h and 8 h) and was analyzed by HPLC (5-95% MeCN/10 mM NH₄HCO₃ in H₂O) (FIG. 7). The 3 peaks in the chromatograph after 30 min of ALP treatment represents compound 5 (peak 1), partially cleaved product (peak 2) and fully cleaved product (peak 3). The reaction almost completes after 6-hour incubation (>95% by integration). The LC trace of the standard dephosphorylated sample 5′ is shown for comparison. Attempts were made to get LC-MS data for 5 but no mass signal was detected. This might result from the poor solubility of 5 in the acidic mobile phase (MeCN/H₂O, 0.1% FA).

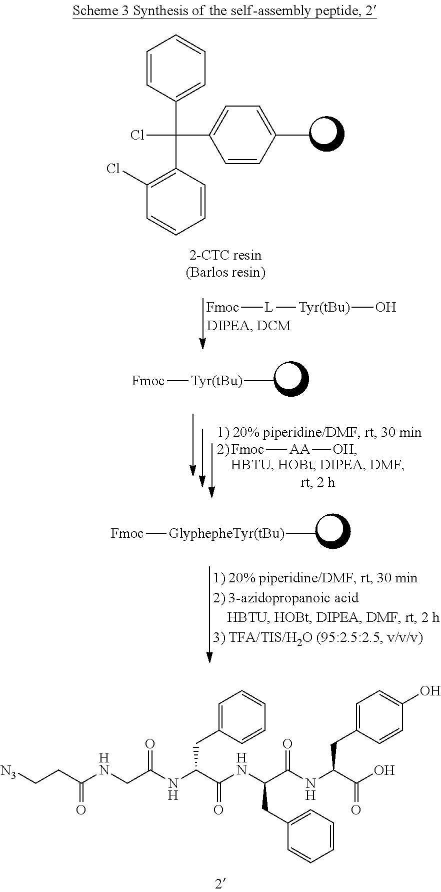

The previous results indicate that aggregation forms upon enzymatic treatment when the concentration of 5 is above 1 μM. The dephosphorylated 5 (5′) was synthesized to study the aggregation in sub-micromolar region. The tetrapeptide derivative 2′ was first synthesized following the standard solid phase peptide synthesis protocol using 2-CTC resin (Scheme 3).

Then 2′ was installed on 1 via copper-catalyzed click chemistry. The reaction mixture was simply washed with water to remove the copper salts. Tetra-n-butylammonium fluoride was added to the crude for TIPS deprotection to give compound 4′. Then, a PEG₂₄ linker was attached to 4′ to yield 5′ via copper-catalyzed click chemistry. (Scheme 2).

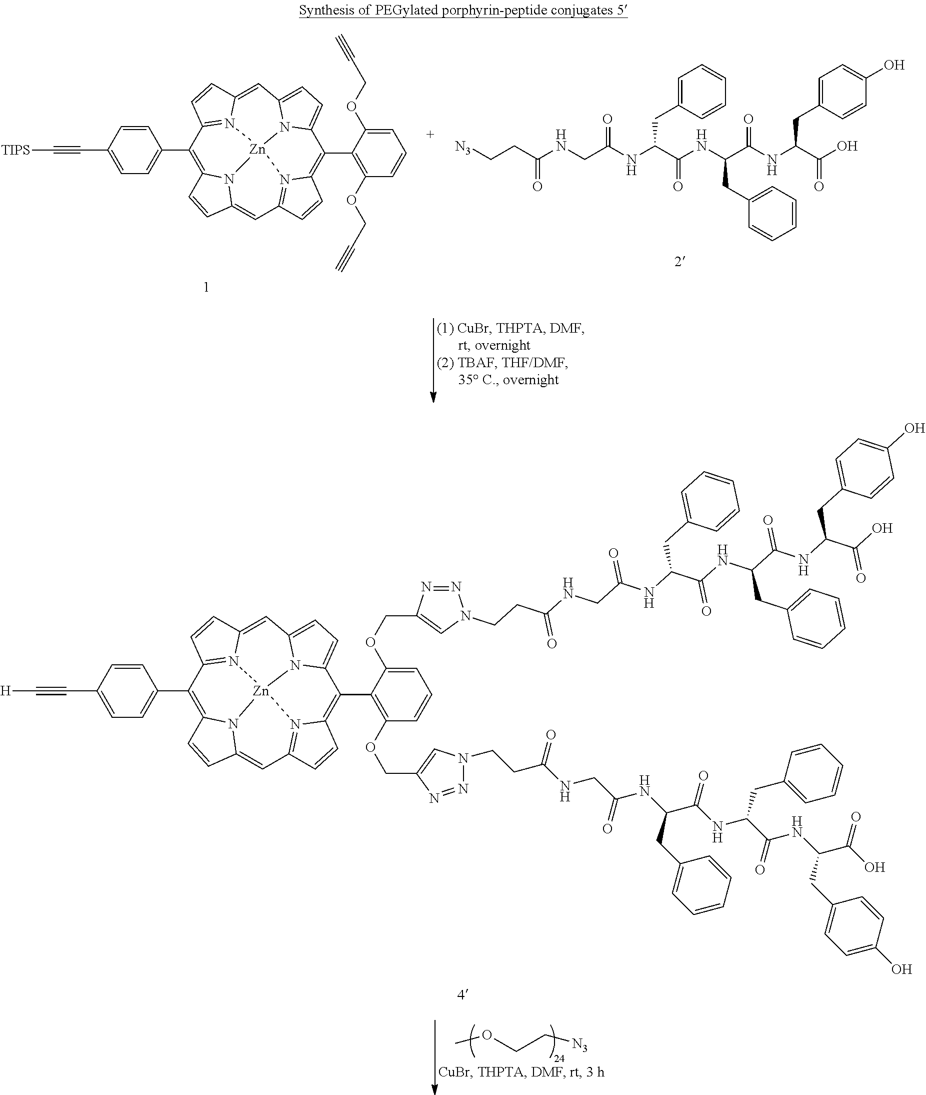

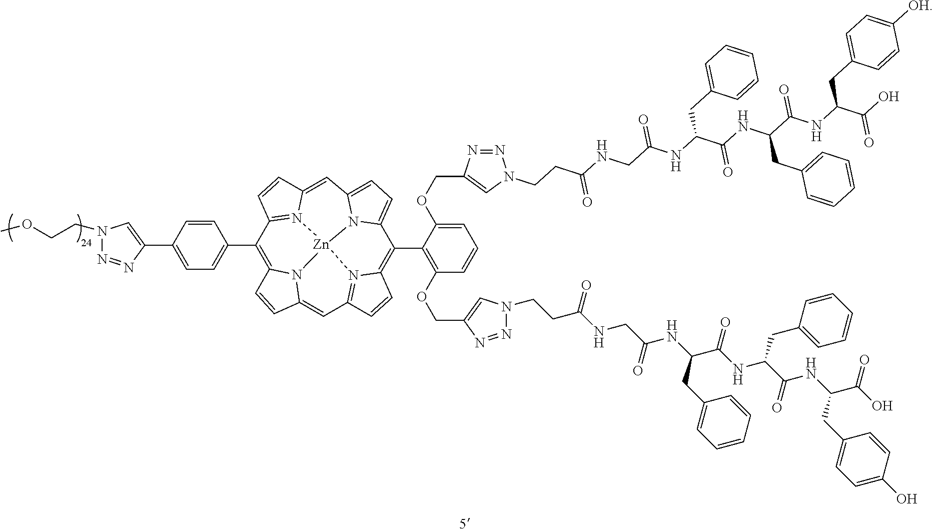

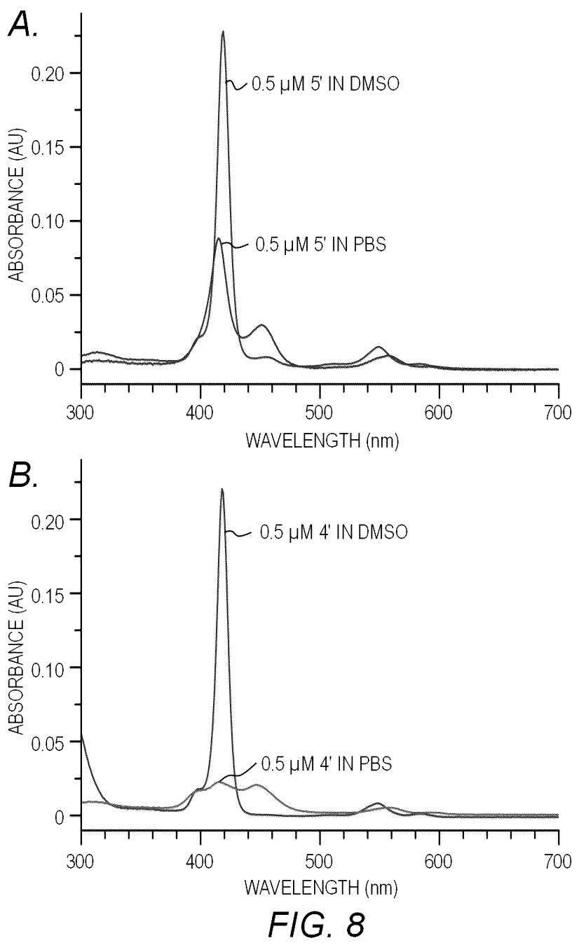

Samples of 5′ at 1 μM in DMSO or PBS were analyzed by absorption spectroscopy (FIG. 8, panel A). The sample in DMSO had the Soret band at 419 nm and showed no sign of aggregation. In PBS, two broad peaks at 417 nm and 455 nm were observed, indicating aggregation of the sample at 1 μM. Samples of 4′ at 1 μM in DMSO or PBS were also analyzed for comparison (FIG. 8, panel B). The spectrum of the sample in DMSO was identical with 5′, while in PBS the Soret band spilt into 3 broad peaks at 409 nm, 419 nm and 455 nm. The different spectra between 4′ and 5′ might result from different ways of porphyrin stacking, as 4′ doesn't have the long PEG chain in its structure.

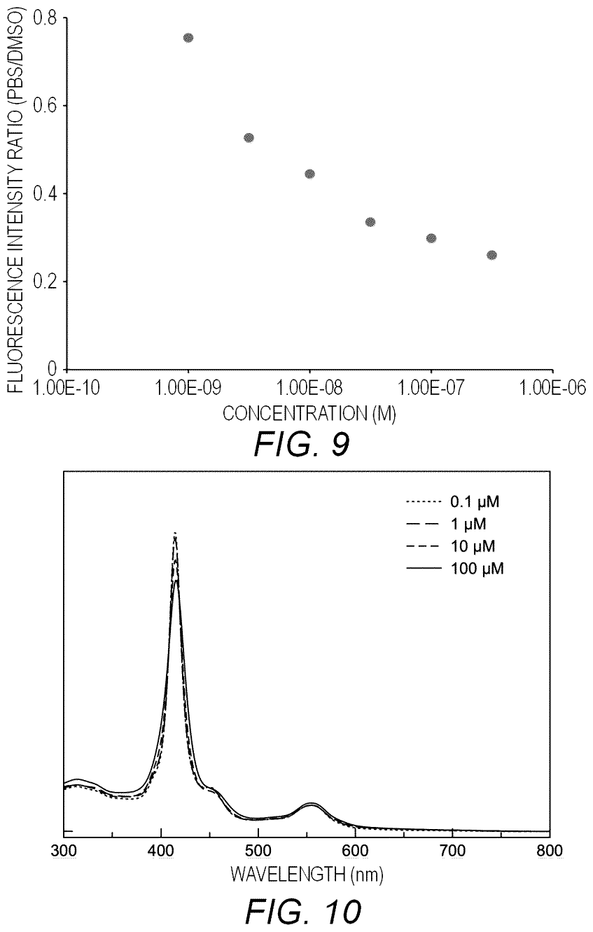

Aggregation of 5′ at low concentrations was analyzed by fluorescence spectroscopy. The ratio of maximum fluorescence intensity of samples in PBS to that of sample in DMSO was plotted against concentration (FIG. 9). The ratio decreased as the concentration increased in the range of 1.0 nM to 316 nM, which implies that 5′ aggregates more in PBS as the concentration increases. Even with the concentration of 316 nM, the sample didn't aggregate completely in PBS and retained ˜30% fluorescence. The long PEG chain in 5′ provides the compound with good hydrophilicity and may contribute to incomplete aggregation. Fluorescence intensity at higher or lower concentrations was not measured due to the detection range of the instrument. The porphyrin-peptide conjugate without PEG chain, 4′, was examined by fluorescence intensity as well. The ratios of maximum fluorescence intensity of sample in PBS to that of sample in DMSO were listed in Table 3. Samples at 31.6 nM, 100 nM, and 316 nM were analyzed and the fluorescence intensity ratio of all samples was below 2%. The results imply that 4′ almost aggregates completely even at a very low concentration.

TABLE 3
The ratio of the fluorescence intensity of 4′ in PBS to the fluorescence
intensity of 4′ in DMSO at different concentrations.

| | |
|---|---|
| Concentration | Intensity Ratio^{a} (PBS/DMSO) |

| | | |
|---|---|---|
| 31.6 | nM | 0.0196 |
| 100 | nM | 0.0133 |
| 316 | nM | 0.0131 |

^{a}Intensity ratio = maximum fluorescence intensity of 4′ in DMSO/maximum fluorescence intensity of 4′ in PBS. Samples were excited at 419 nm. The maximum fluorescence intensity was recorded at 641 nm for DMSO solution and 645 nm for PBS solution. The maximum fluorescence intensity was recorded at 641 nm for samples in DMSO and 645 nm for samples in PBS [Ex/Em bandpass = 10 nm, integration time = 0.05 s]. No fluorescence was detected for sample in PBS at a concentration lower than 31.6 nM.

The spectra of 5 at concentrations over a 1000-fold range (all in PBS) are shown in FIG. 10. Samples of 0.1 μM, 1 μM, 10 μM and 100 μM were analyzed using cuvettes with a pathlength of 10 cm, 1 cm, 0.1 cm and 0.01 cm, respectively. There is only a very slight broadening of the absorption bands at 100 μM, and at all concentrations the general spectroscopic features characteristic of the porphyrin are retained. The result indicates that 5 is soluble in PBS at 100 μM with essentially no aggregation or assembly.

### CONCLUSIONS

PEGylated porphyrin-peptide conjugate, 5, was synthesized and the in vitro enzymatically triggered assembly test was performed with compound 5 and ALP. The results of absorption spectroscopy indicate that the 5 dephosphorylated and aggregated in PBS upon the treatment of ALP. The aggregation was reversible upon the addition of DMSO. Based on the results in hand, the aggregation requires the lowest substrate concentration of 1.0 μM.

Further studies with the dephosphorylated porphyrin-peptide conjugate 5′ reveal that the aggregation can still form in sub-micromolar region. The reason why 5 did not aggregate upon treatment of ALP at low concentration may be due to the low efficiency of enzyme cleavage with low concentration of substance. The study on 4′ implies that the lower concentration of aggregation can be achieved by altering the PEG linker.

Different porphyrin species showed different absorption spectra upon aggregation. The Soret band split into 2 peaks for 5 and 5′ and 3 peaks for 4′. It's possible that the absence of a long PEG linker in 4′ could result in differences in the way it stacks compared to other molecules with PEG linkers. How the porphyrin-peptide conjugates interact with each other and affect the absorption remains unclear. Efforts were made to characterize the size of the aggregates by dynamic light scattering (DLS) but no data with good quality was obtained. The size data presented by DLS showed a wide size distribution. It's possible that the aggregates have non-spherical shapes (for example, fibril shape) which make the sample not suitable for DLS analysis. The flexibility of the long PEG linker may cause the irregular shapes of the aggregates.

It was demonstrated that, at the very low concentration of 0.1 μM, the activated form of 5 came out of solution without forming a gel superstructure. Without wishing to be bound to any particular theory, the activated compound may not require interaction with another hydrogelator to promote retention in the target tissue, which may allow the compound to concentrate in a tissue by its mere insolubility in interstitial fluid and/or be used for treatment schemes that rely on administration of a drug to a body cavity, for example, intra-peritoneal drug delivery for ovarian cancer which has metastasized to the omentum and/or intercranial administration following tumor resection.

In a previous study (Yao, 2008), a phosphorylated porphyrin P1-(PO₄)₂ was synthesized, and the assembly of the molecule was tested upon the treatment of the shrimp alkaline phosphatase. P1-(PO₄)₂ was found to exhibit satisfactory solubility in water (>1 mM), but formed microcrystalline precipitates upon shrimp alkaline phosphatase treatment. A crippling deficiency of the P1-(PO₄)₂ compounds was decomposition upon standing, whereupon the phospho moiety was lost and the resulting hydroxy-substituted swallowtail porphyrin underwent self-aggregation and precipitation. The premature cleavage was attributed to intramolecular coordination of the phospho unit at the apical site of the metal chelate of the macrocycle. Accordingly, the studies of the porphyrins in Yao et al. largely concerned free base macrocycles, not metal chelates. The enzymatic cleavage of P1-(PO₄)₂ and analogues was not studied with metal chelates and at neutral pH owing to concerns about spontaneous, intramolecular-facilitated loss of the phospho moieties. Compared to P1-(PO₄)₂, the new phosphate-peptide conjugate 5 was improved in the following ways.

(1) For the enzyme test, compound 5 was tested under physiological conditions (in PBS at pH 7.4) whereas P1-(PO₄)₂ was examined in Tris-HCl buffer (pH 9.0).
(2) The concentration of P1-(PO₄)₂ in the enzyme test was 68 μM, which greatly exceeds that for clinical use. In contrast, the assembly test of 5 or 5′ was carried out in the sub-micromolar region. The two compounds 5 or 5′ assembled in these low concentrations following enzymatic treatment; thus demonstrating that compounds 5 and 5′ can work in a therapeutic regimen.
(3) Compound P1-(PO₄)₂ formed microcrystals upon enzyme treatment. In contrast, compound 5 did not form microcrystals or precipitates upon ALP treatment even at a concentration as high as 200 μM. Instead, 5 formed a smooth hydrogel assembly that was devoid of microcrystals and precipitates. No precipitation or microcrystals were observed with 5 after high-speed centrifugation (>10000 rpm) or long-time storage (>1 month).

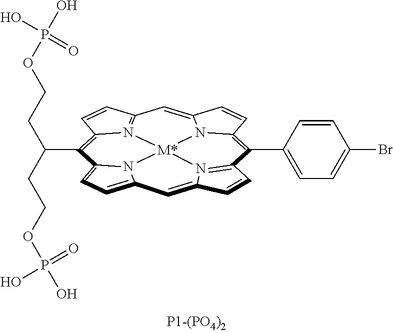

### Materials

Porphyrin 1 was prepared by standard methods for accessing trans-AB-porphyrins (Fan et al., Tetrahedron 2005, 61, 10291-10302). All Fmoc-protected amino acids and 2-CTC resin were purchased from Chemimpex. TBAF (1 M in THF) was purchased from Oakwood Chemicals. mPEG₂₄-N₃ was purchased from Broadpharm. Alkaline phosphatase from bovine intestinal mucosa was purchased from Sigma-Aldrich.

### Experimental Section

Peptide synthesis (2 and 2′). Peptides were synthesized following standard Fmoc solid phase peptide synthesis using 2-chlorotrityl chloride resin (1.43 mmol/g) and the corresponding Fmoc-protected amino acids. Briefly, the first amino acid, Fmoc-L-phosphotyrosine (1.5 equiv) was loaded onto the resin in CH₂Cl₂/DMSO (19:1 v/v) with DIPEA (2.0 equiv) as the base. After loading the first amino acid to the resin, methanol was used to ensure that all the active sites of the resin were protected. The resin was treated with 20% piperidine in DMF for 30 min to remove the Fmoc group. The following amino acid couplings were performed in DMF using the Fmoc-protected amino acid (3.0 equiv), HOBt (3.0 equiv) and HBTU (3.0 equiv) as the coupling reagents, and DIPEA (6.0 equiv) as the base. After the installation of the last amino acid, 3-azidopropanoic acid was installed following the same coupling protocol. The peptide derivative was cleaved using 95% TFA, 2.5% TIS, and 2.5% H₂O for 1 h. Then ice-cold ethyl ether was then added to precipitate the crude peptides. The crude was further purified by preparative HPLC [Buchi® PrepPure C850, Buchi® column (10 μm C18 100 Å, LC Column 150×10 mm), linear gradient with 30-80% MeCN/H₂O, 0.1% TFA].

RH16-GffY(p)-OH (3). CuBr (4.8 mg, 33.8 μmol) and Tris(benzyltriazolylmethyl)amine (29.4 mg, 67.6 μmol) were dissolved in DMSO (50 μL) first and added to a solution of 1 (5.5 mg, 6.8 μmol) and 2 (12.0 mg, 16.9 μmol) in DMSO (450 μL). The mixture was stirred overnight at room temperature. After removing DMSO with a lyophilizer, the reaction mixture was suspended in water. The saturated NH₄Cl solution was added dropwise to precipitate the crude. The crude was collected by filtering with a 0.1 μm filter membrane. No further purification was performed. MALDI-MS obsd 2232.85, calcd 2232.72 [(M+H)⁺, M=C₁₁₃H₁₁₆N₁₈O₂₂P₂SiZn].

Deprotected RH16-GffY(p)-OH (4). A solution of TBAF (8.7 μL, 1 M in THF) was added to the crude of 3 (6.5 mg, 2.9 μmol) in DMSO (250 μL). The reaction was stirred at room temperature for 5 h. After removing DMSO with a lyophilizer, the reaction mixture was suspended in water. The saturated NH₄Cl solution was added dropwise to precipitate the crude. The crude was collected by filtered with a 0.1 μm filter membrane and washed twice with 2 mL of water. The crude on the filter membrane was dissolved with DMSO and was immediately used for the next step. MALDI-MS obsd 2074.51, calcd 2074.57 (M⁺, M=C₁₀₄H₉₆N₁₈O₂₂P₂Zn).

Deprotected RH16-GffY-OH (4′). CuBr (2.1 mg, 14.4 μmol) and Tris(benzyltriazolylmethyl) amine (12.5 mg, 28.8 μmol) were dissolved in DMF (50 μL) first and added to a solution of 1 (5.9 mg, 7.2 μmol) and 2′ (10.0 mg, 15.9 μmol) in DMF (450 μL). The mixture was stirred overnight at room temperature. The reaction mixture was precipitated with 20 mL cold ethyl ether. The precipitate was washed 3 times with water and dried under high vacuum. The crude was dissolved in 500 μL of DMF and 22 μL of TBAF/THF (1 M, 22 μmol) was added. The reaction was stirred overnight at 35° C. Then the mixture was precipitated again with ethyl ether and washed 3 times with ether ethyl followed by water. No further purification was performed. MALDI-MS obsd 1914.59, calcd 1914.64 (M⁺, M=C₁₀₄H₉₄N₁₈O₁₆Zn).

PEG24-GffY(p)-OH (5). CuBr (1.0 mg, 7.3 μmol) and Tris(benzyltriazolylmethyl) amine (6.5 mg, 14.6 μmol) were dissolved in DMSO (50 L) first and added to a solution of 4 (crude, 6.0 mg, 2.9 μmol) and mPEG24-N₃ (4.0 mg, 3.6 μmol) in DMSO (300 μL). The mixture was stirred overnight at room temperature. After removing DMSO with a lyophilizer, the reaction mixture was suspended in water. The saturated NH₄Cl solution was added dropwise to precipitate the crude. The crude was collected by filtering with a 0.1 μm filter membrane. The crude was then purified by pTLC [C18 reverse-phase, THF/H₂O (5:5)]. The sample was homogeneous by RP-HPLC. MALDI-MS obsd 3188.34, calcd 3188.23 (M⁺, M=C₁₅₃H₁₉₅N₂₁O₄₆P₂Zn).

PEG24-GffY-OH (5′). CuBr (0.60 mg, 4.2 μmol) and Tris(benzyltriazolylmethyl) amine (3.7 mg, 8.4 μmol) were dissolved in DMF (50 μL) first and added to a solution of 4′ (crude, 4.0 mg, 2.1 μmol) and mPEG24-N₃ (2.6 mg, 2.3 μmol) in DMF (450 μL). The mixture was stirred for 3 h at room temperature. The reaction mixture was precipitated with 20 mL cold ethyl ether. The crude was purified by pTLC [C18 reverse-phase, THF/H₂O (6:4)]. The sample was homogeneous by RP-HPLC. MALDI-MS obsd 3029.05, calcd 3028.30 [(M+H)⁺, M=C₁₅₃H₁₉₃N₂₁O₄₀0Zn).

Enzymatic assay. The stock solution of 5 in DMSO (2 mM) was diluted with PBS to prepare sample solutions with different concentrations. The final concentrations of 5 in each sample were 0.20 μM, 0.50 μM, 1.0 μM, 2.0 μM, 5.0 μM and 10 μM, respectively. A solution of alkaline phosphatase from bovine intestinal mucosa (10 μL, 100 U/mL in PBS) was added to each sample solution to reach a final volume of 500 μL (PBS containing 0.5% DMSO). The reaction mixture was incubated overnight at 37° C. Then each sample was analyzed by absorption spectroscopy. Samples without the treatment of ALP were also analyzed to serve as controls.

To confirm the aggregation of the dephosphorylated 5 in PBS, 500 μL of DMSO was added to one of the treated samples (10 μM) and was then analyzed by absorption spectroscopy.

Fluorescence spectroscopy. Samples were dissolved in PBS and DMSO at different concentrations. The excitation wavelength was 419 nm for all samples in DMSO, 415 nm for 5′ in PBS, and 419 nm for 4′ in PBS. The intensity ratio was calculated with the maximum emission of each sample. The excitation and emission bandpasses were both 10 nm and the integration time was 0.05 s.

Reverse phase high performance liquid chromatography. A solution of 200 μM 5 in PBS was treated with 2 U/mL ALP and incubated at 37° C. A small part of sample was taken from the reaction at different time points (30 min, 1 h, 2 h, 4 h, 6 h and 8 h) and was analyzed by RP-HPLC with an Agilent Zorbax SB-C18 column (5 μm, 4.6×150 mm). The sample was run for 15 min with a linear gradient (5-95% MeCN/10 mM NH₄HCO₃ in H₂O) and detected with a diode-array detector at 420 nm.

### REFERENCES

Du, X.; Zhou, J.; Wang, H.; Shi, J.; Kuang, Y.; Zeng, W.; Yang, Z.; Xu, B. In Situ Generated D-Peptidic Nanofibrils as Multifaceted Apoptotic Inducers to Target Cancer Cells. Cell Death Dis. 2017, 8 e2614.
Shi, J.; Du, X.; Yuan, D.; Zhou, J.; Zhou, N.; Huang, Y.; Xu, B. D-Amino Acids Modulate the Cellular Response of Enzymatic-Instructed Supramolecular Nanofibers of Small Peptides. Biomacromolecules 2014, 15 (10), 3559-3568.
Wang, T. T.; Xia, Y. Y.; Gao, J. Q.; Xu, D. H.; Han, M. Recent Progress in the Design and Medical Application of in Situ Self-Assembled Polypeptide Materials. Pharmaceutics 2021, 13 (5), 1-19.
Wei, H.; Min, J.; Wang, Y.; Shen, Y.; Du, Y.; Su, R.; Qi, W. Bioinspired Porphyrin-Peptide Supramolecular Assemblies and Their Applications. J. Mater. Chem. B 2022, 10 (45), 9334-9348.
Yao, Z.; Borbas, K. E.; Lindsey, J. S. Soluble Precipitable Porphyrins for Use in Targeted Molecular Brachytherapy. New J. Chem. 2008, 32 (3), 436-451.
Zou, Q.; Abbas, M.; Zhao, L.; Li, S.; Shen, G.; Yan, X. Biological Photothermal Nanodots Based on Self-Assembly of Peptide-Porphyrin Conjugates for Antitumor Therapy. J. Am. Chem. Soc. 2017, 139 (5), 1921-1927.
Cao, Z.; Li, W.; Liu, R.; Li, X.; Li, H.; Liu, L.; Chen, Y.; Lv, C.; Liu, Y. PH- and Enzyme-Triggered Drug Release as an Important Process in the Design of Anti-Tumor Drug Delivery Systems. Biomed. Pharmacother. 2019, 118, 109340.
Chakravarty, P. K.; Carl, P. L.; Weber, M. J.; Katzenellenbogen, J. A. Biological Activity of Peptidylacivicin and Peptidylphenylenediamine Mustard. J. Med. Chem. 1983, 26, 633-638.
Cheng, Y. J.; Qin, S. Y.; Liu, W. L.; Ma, Y. H.; Chen, X. S.; Zhang, A. Q.; Zhang, X. Z. Dual-Targeting Photosensitizer-Peptide Amphiphile Conjugate for Enzyme-Triggered Drug Delivery and Synergistic Chemo-Photodynamic Tumor Therapy. Adv. Mater. Interfaces 2020, 7 (19), 1-9.
De Groot, F. M. H.; Van Berkom, L. W. A.; Scheeren, H. W. Synthesis and Biological Evaluation of 2′-Carbamate-Linked and 2′-Carbonate-Linked Prodrugs of Paclitaxel Selective Activation by the Tumor-Associated Protease Plasmin. J. Med. Chem. 1999, 42 (25), 5277-5283.
Denmeade, S. R.; Nagy, A.; Gao, J.; Lilja, H.; Schally, A. V.; Isaacs, J. T. Enzymatic Activation of a Doxorubicin-Peptide Prodrug by Prostate-Specific Antigen. Cancer Res. 1998, 58 (12), 2537-2540.
Kalafatovic, D.; Nobis, M.; Javid, N.; Frederix, P. W. J. M.; Anderson, K. I.; Saunders, B. R.; Ulijn, R. V. MMP-9 Triggered Micelle-to-Fibre Transitions for Slow Release of Doxorubicin. Biomater. Sci. 2015, 3 (2), 246-249.
Li, D.; Lu, B.; Huang, Z.; Xu, P.; Zheng, H.; Yin, Y.; Xu, H.; Liu, X.; Chen, L.; Lou, Y.; Zhang, X.; Xiong, F. A Novel Melphalan Polymeric Prodrug: Preparation and Property Study. Carbohydr. Polym. 2014, 111, 928-935.
Li, H.; Wang, P.; Deng, Y.; Zeng, M.; Tang, Y.; Zhu, W. H.; Cheng, Y. Combination of Active Targeting, Enzyme-Triggered Release and Fluorescent Dye into Gold Nanoclusters for Endomicroscopy-Guided Photothermal/Photodynamic Therapy to Pancreatic Ductal Adenocarcinoma. Biomaterials 2017, 139, 30-38.
Raposo Moreira Dias, A.; Pina, A.; Dean, A.; Lerchen, H. G.; Caruso, M.; Gasparri, F.; Fraietta, I.; Troiani, S.; Arosio, D.; Belvisi, L.; Pignataro, L.; Dal Corso, A.; Gennari, C. Neutrophil Elastase Promotes Linker Cleavage and Paclitaxel Release from an Integrin-Targeted Conjugate. Chem. Eur. J. 2019, 25 (7), 1696-1700.
Sun, L.; Xie, S.; Qi, J.; Liu, E.; Liu, D.; Liu, Q.; Chen, S.; He, H.; Yang, V. C. Cell-Permeable, MMP-2 Activatable, Nickel Ferrite and His-Tagged Fusion Protein Self-Assembled Fluorescent Nanoprobe for Tumor Magnetic-Targeting and Imaging. ACS Appl. Mater. Interfaces 2017, 9 (45), 39209-39222.
Tanaka, A.; Fukuoka, Y.; Morimoto, Y.; Honjo, T.; Koda, D.; Goto, M.; Maruyama, T. Cancer Cell Death Induced by the Intracellular Self-Assembly of an Enzyme-Responsive Supramolecular Gelator. J. Am. Chem. Soc. 2015, 137 (2), 770-775.
Zhang, Z. T.; Huang-Fu, M. Y.; Xu, W. H.; Han, M. Stimulus-Responsive Nanoscale Delivery Systems Triggered by the Enzymes in the Tumor Microenvironment. Eur. J. Pharm. Biopharm. 2019, 137, 122-130.
Franciscus et al., Synthesis and Biological Evaluation of Novel Prodrugs of Anthracyclines for Selective Activation by the Tumor-Associated Protease Plasmin. J. Med. Chem. 1999, 42 (25) 5277-5283.

1. A compound comprising:
a tetrapyrrole macrocycle comprising a radionuclide;
a hydrogelator attached to the tetrapyrrole macrocycle;
a water solubilizing group attached to the hydrogelator; and
a cleavage site that is between the hydrogelator and the water solubilizing group.

2. The compound of claim 1, wherein the radionuclide is ⁶⁴Cu, ⁶⁷Cu, ⁴⁴Sc, ⁴⁷Sc, ⁶⁷Ga, ⁶⁸Ga, ⁸⁹Zr, ^{99m}Tc, ¹¹¹In, ¹⁷⁷Lu, ⁵¹Mn, ^{52g}Mn, ^{52m}Mn, ⁸⁶Y, ⁶²Zn, ⁵⁷Co, ¹²³I, ¹²⁵I, ¹³¹I, ¹⁸F, or ²¹¹At.

3. The compound of claim 1, wherein the hydrogelator comprises a first peptide having a sequence of FFY, GGGH (SEQ ID NO:1), ILQINS(SEQ ID NO:2), FKFE (SEQ ID NO:3), FKFEFKFE (SEQ ID NO: 4), QQKFQFQFEQQ (SEQ ID NO:5), or KKFKFEFEF (SEQ ID NO:6).

4. The compound of claim 1, further comprising a second peptide having a sequence of GFLG (SEQ ID NO: 7), (GG), wherein n is between 2-4 (SEQ ID NO:8), CRQAGFSL (SEQ ID NO:9), PLGVR (SEQ ID NO:10), PLGL (SEQ ID NO:11), FFAGLAG (SEQ ID NO:12), HSSKLQ (SEQ ID NO:13), aFK wherein a is D-alanine, or NPA, and the cleavage site is within or after the second peptide.

5. The compound of claim 1, wherein the cleavage site is within the hydrogelator.

6.-7. (canceled)

8. The compound of claim 1, wherein the water solubilizing group comprises a polyethylene glycol (PEG), sulfonate, ammonium, carboxylate, betaine, phosphate, phosphonate, amino acid residue, glycoside, and/or peptide.

9. (canceled)

10. The compound of claim 1, wherein the tetrapyrrole macrocycle is a porphyrin.

11.-16. (canceled)

17. The compound of claim 1, wherein the compound comprises two or more hydrogelators, cleavage sites, and/or water solubilizing groups.

18. (canceled)

19. The compound of claim 1, wherein the tetrapyrrole macrocycle comprises a swallowtail group and the hydrogelator, cleavage site, and/or water solubilizing group is bound to an atom of the swallowtail group.

20. The compound of claim 19, wherein the swallowtail group has a structure of:
—CH((CH₂)ₙ₂(C(O)O)ₘ₂(CH₂)ₚ₂Y)₂,
wherein:
n2 is an integer of 1 to 20;
m2 is 0 or 1;
p2 is 0 or an integer of 1 to 10; and
Y is absent, —OCH₂—, —C(O)— or —C(O)O—.

21. The compound of claim 19, wherein the swallowtail group has a structure of:
—CH((CH₂)ₙ₃(OCH₂CH₂)ₘ₃X(CH₂)ₚ₃Y)₂,
wherein:
n3 is an integer of 1 to 20;
m3 is 0 or 1 to 24;
X is oxygen or absent;
p3 is 0 or an integer of 1 to 10; and
Y is absent, —C(O)O—, —OCH₂—, —O—, —C(O)—, —PO₃H—, —SO₃—, or —OCH₂C(O)O—.

22. The compound of claim 19, wherein the swallowtail group has a structure of:
—CH((CH₂)ₙ₄(C(O)NH)(CH₂)ₘ₄(OCH₂CH₂)ₚ₄Y)₂,
wherein:
n4 is an integer of 1 to 20;
m4 is 1 to 20;
p4 is 0 or an integer of 1 to 24; and
Y is absent, —C(O)O—, —OCH₂—, —O—, —C(O)—, —PO₃H—, —SO₃—, or —OCH₂C(O)O—.

23. The compound of claim 1, wherein the tetrapyrrole macrocycle comprises an aryl moiety and the hydrogelator, cleavage site, and/or water solubilizing group is bound to an atom of the aryl moiety.

24.-31. (canceled)

32. The compound of claim 1, further comprising a first linker, wherein the first linker is attached to the tetrapyrrole macrocycle and the hydrogelator.

33. The compound of claim 1, wherein the tetrapyrrole macrocycle has a structure of Formula Ia or Formula Ib 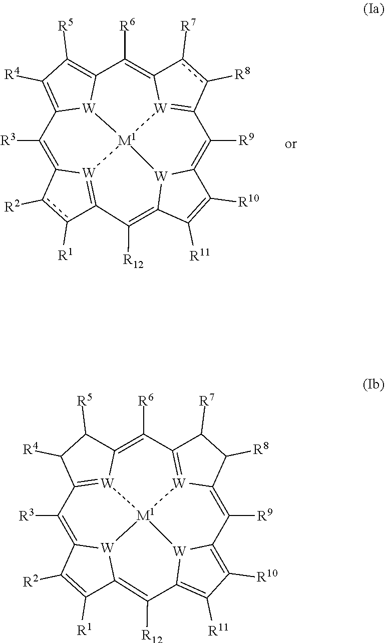
wherein: [symbol]
indicates a single bond or a double bond;
R¹, R², R³, R⁴, R⁵, R⁶, R⁷, R⁸, R⁹, R¹⁰, R¹¹, and R¹² are each independently selected from the group consisting of a hydrogen, alkyl, alkenyl, alkynyl, cycloalkyl, cycloalkylalkyl, cycloalkylalkenyl, cycloalkylalkynyl, heterocyclo, heterocycloalkyl, heterocycloalkenyl, heterocycloalkynyl, aryl, aryloxy, arylalkyl, arylalkenyl, arylalkynyl, heteroaryl, heteroarylalkyl, heteroarylalkenyl, heteroarylalkynyl, alkoxy, halo, mercapto, azido, cyano, formyl, carboxylic acid, hydroxyl, nitro, acyl, alkylthio, amino, alkylamino, arylalkylamino, disubstituted amino, acylamino, acyloxy, ester, amide, sulfoxyl, sulfonyl, sulfonate, sulfonic acid, sulfonamide, urea, alkoxylacylamino, aminoacyloxy, hydrophilic groups, linking groups, bioconjugatable groups, surface attachment groups, targeting groups, the hydrogelator, the cleavage site, and/or the water solubilizing group, each of which may optionally be substituted;
or R¹ and R² together represent a fused aromatic or heteroaromatic ring system that is substituted or unsubstituted;
or R² and R³ together represent a fused aromatic or heteroaromatic ring system that is substituted or unsubstituted;
or R³ and R⁵ together represent a fused aromatic or heteroaromatic ring system that is substituted or unsubstituted;
or R⁴ and R⁵ together represent a fused aromatic or heteroaromatic ring system that is substituted or unsubstituted;
or R⁴ and R⁷ together represent a fused aromatic or heteroaromatic ring system that is substituted or unsubstituted;
or R⁷ and R⁸ together represent a fused aromatic or heteroaromatic ring system that is substituted or unsubstituted;
or R⁹ and R¹⁰ together represent a fused aromatic or heteroaromatic ring system that is substituted or unsubstituted; or
or R¹⁰ and R¹¹ together represent a fused aromatic or heteroaromatic ring system that is substituted or unsubstituted; and
M¹ is the radionuclide, and
W is N, O, S, Se, or CH; and
wherein at least one of R¹, R², R³, R⁴, R⁵, R⁶, R⁷, R⁸, R⁹, R¹⁰, R¹¹, and R¹² comprises the hydrogelator, cleavage site, and water solubilizing group or is substituted with the hydrogelator, cleavage site, and water solubilizing group.

34. The compound of claim 1, further comprising a targeting agent.

35. The compound of claim 34, further comprising a second linker, wherein the second linker is attached to the tetrapyrrole macrocycle and the targeting agent.

36. A method of diagnosing a disease or disorder in a subject, the method comprising:
administering the compound of claim 1 to the subject; and
imaging the subject and/or detecting the compound, thereby diagnosing the disease or disorder in the subject.

37. (canceled)

38. A method of treating a subject in need thereof, the method comprising:
administering the compound of claim 1 to the subject, thereby treating the subject.

39.-42. (canceled)

43. A method of aggregating and/or immobilizing a radionuclide in a subject, the method comprising:
administering the compound of claim 1 to the subject, thereby aggregating and/or immobilizing the radionuclide in the subject.

44.-47. (canceled)


SEQUENCE LISTING (13 sequences)

SEQ ID NO: 1 - protein, 4 aa, synthetic construct
  LIPID 1: note = palmitoyl
GGGH

SEQ ID NO: 2 - protein, 6 aa, synthetic construct
ILQINS

SEQ ID NO: 3 - protein, 4 aa, synthetic construct
FKFE

SEQ ID NO: 4 - protein, 8 aa, synthetic construct
FKFEFKFE

SEQ ID NO: 5 - protein, 11 aa, synthetic construct
QQKFQFQFEQQ

SEQ ID NO: 6 - protein, 9 aa, synthetic construct
KKFKFEFEF

SEQ ID NO: 7 - protein, 4 aa, synthetic construct
GFLG

SEQ ID NO: 8 - protein, 8 aa, synthetic construct
  VARIANT 5..8: note = Residues are present or absent
GGGGGGGG

SEQ ID NO: 9 - protein, 8 aa, synthetic construct
CRQAGFSL

SEQ ID NO: 10 - protein, 5 aa, synthetic construct
PLGVR

SEQ ID NO: 11 - protein, 4 aa, synthetic construct
PLGL

SEQ ID NO: 12 - protein, 7 aa, synthetic construct
  VARIANT 6..7: note = Residues may be any other amino acid, preferably G, P, V, L, K, or D
FFAGLAG

SEQ ID NO: 13 - protein, 6 aa, synthetic construct
HSSKLQ